(19) United States
(12) Patent Application Publication
(10) Pub. No.: US 2011/0280839 A1
(43) Pub. Date: Nov. 17, 2011

(54) Entomopathogenic Fungi and Uses Thereof

(75) Inventor: Stephen Reynold Ford, Bombay (NZ)

(73) Assignee: PWC Tower, Auckland (NZ)

(21) Appl. No.: 13/124,273

(22) PCT Filed: Oct. 9, 2009
(86) PCT No.: PCT/NZ09/00217

§ 371 Date: Aug. 5, 2011

Related U.S. Application Data
(60) Provisional application No. 61/105,092, filed on Oct. 14, 2008.
(60) Provisional application No. 61/234,028, filed on Aug. 14, 2009.

(51) Int. Cl. A01N 63/04; A01P 7/04; C12N 1/14

(52) U.S. Cl. 424/93.3; 435/254.1; 424/93.5

(57) ABSTRACT

The present invention provides a strain of entomopathogenic Beauveria bassiana, compositions comprising the entomopathogenic fungi strain or metabolites of the strain, and the use of the entomopathogenic fungi strain and compositions as biological control agents. Methods for the biological control of phytopathogenic insects using an entomopathogenic Beauveria bassiana fungi strain or one or more metabolites thereof, optionally together with other entomopathogenic fungi including fungi selected from strains of Lecanicillium spp., Paecilomyces fumosoroseus, and compositions comprising said fungi or metabolites thereof are also provided.

### FIELD OF THE INVENTION

This invention relates to entomopathogenic fungi and metabolites thereof, compositions comprising said entomopathogenic fungi or one or more metabolites thereof, and the use of such entomopathogenic fungi and compositions as biological control agents. Methods for the biological control of phytopathogenic insects, including aphids, thrips, white fly, mealy bug, and the like using the entomopathogenic fungi, Beauveria bassiana and compositions comprising said fungi or one or more metabolites thereof are also provided.

### BACKGROUND OF THE INVENTION

Plant disease caused by pathogens such as insects are a significant economic cost to plant based agriculture and industries. Losses may arise through spoilage of produce both pre and post harvest, loss of plants themselves, or through reduction in growth and production abilities.

Traditionally, control of plant pathogens has been pursued through the application of chemical insecticides. The use of chemicals is subject to a number of disadvantages. The pathogens can and have developed tolerance to chemicals to over time, producing resistant populations. Indeed, resistance to pesticides is the greatest challenge to the viability of the horticultural industry.

The problem is particularly illustrated with reference to a number of economically important phytopathogenic insects. Populations of western flower thrips worldwide are reported to be resistant to most groups of pesticides including the following examples; acephate, abamectin, chlorpyrifos, endosulfan, methomyl, methiocarb, omethoate, pyrazophos and tau-fluvalinate. Populations of onion thrips in New Zealand have developed resistance to deltamethrin, and local populations have been reported to be resistance to diazinon and dichlorvos. Onion thrips in the United States have been reported to be resistant to many pesticides (Grossman, 1994). Greenhouse whitefly has reportedly developed resistance to organochlorine, organophosphate, carbamate and pyrethroid insecticides (e.g. Georghiou 1981, Anis & Brennan 1982, Elhag & Horn 1983, Wardlow 1985 and Hommes 1986). Resistance has also been reported in newer insecticides, buprofezin and teflubenzuron (Gorman et al. 2000).

Chemical residues may also pose environmental hazards, and raise health concerns. The revival of interest in biological control such as microbial insecticides over the last 20 years has come directly from public pressure in response to concerns about chemical toxicities. Biological control presents an alternative means of controlling plant pathogens which is potentially more effective and specific than current methods, as well as reducing dependence on chemicals. Such biological control methods are perceived as a “natural” alternative to insecticides with the advantage of greater public acceptance, reduced environmental contamination, and increased sustainability.

Mechanisms of biological control are diverse. One mechanism which has been demonstrated to be effective is the use of antagonistic microorganisms such as bacteria to control phytopathogenic insects. For example, the large scale production of Bacillus thuringiensis enabled the use of this bacterio-insecticide to control painted apple moth in Auckland, New Zealand.

There is little information on the successful application of entomopathogenic fungi and their industrial production is still relatively unsophisticated. Applications of entomopathogenic fungi as biological control agents (BCAs) using Lecanicillium muscarium (previously known as Verticillium lecanii), Beauveria bassiana and Metarhizium anisopliae have been developed in the US, Europe, Africa and Russia. However, to date none of the candidates have proved ideal, possibly through a failure to quickly establish and/or survive the environmental variability existing in the field. Indeed, existing candidates do not appear to have met with significant grower acceptance, and may be perceived to be uneconomic.

This is compounded by the frequent unavailability or delay in availability of entomopathogenic fungi developed in one country to the horticulturists of another country, for example due to regulatory constraints. Furthermore, many non-indigenous fungi may be unlikely to be suitable for, or able to survive or flourish in, local conditions.

Surprisingly, the Applicants have now identified and isolated a Beauveria strain not mentioned in any of the earlier reports as an effective BCA. The Applicants have determined that this species is highly effective in controlling phytopathogenic insects, including but not limited to thrips, aphids and whitefly, and in successfully surviving and establishing in the field.

It is therefore an object of the present invention to provide a strain of Beauveria useful in the biological control of phytopathogenic insects, or at least to provide the public with a useful choice.

### SUMMARY OF THE INVENTION

Accordingly, in one aspect the present invention provides a biologically pure culture of Beauveria bassiana fungus strain K4B3 on deposit at National Measurement Institute of Australia (NMIA) under Accession No. V08/025855 deposited 23 Sep. 2008, or a culture having the identifying characteristics thereof.

In a further aspect the present invention provides spores obtainable from a fungus of the invention.

In another aspect, the present invention provides the use of at least the fungi as defined above together with at least one carrier in the preparation of a composition.

In another aspect, the present invention provides the use of spores from at least one fungi as defined above together with at least one carrier in the preparation of a composition.

In one embodiment, said at least one fungi is in a reproductively viable form and amount.

In a further aspect the present invention provides a composition which comprises at least one fungi as defined above together with at least one carrier.

Preferably, said at least one fungi is in a reproductively viable form and amount.

In a further aspect the invention provides a composition comprising spores obtainable from a least one fungi of the invention together with at least one carrier.

Preferably, said composition is a biological control composition, more preferably said biological control composition is an entomopathogenic composition.

Preferably, said biological control composition comprises at least one agriculturally acceptable carrier.

In a further aspect the present invention provides a composition which comprises at least one metabolite of B. bassiana strain K4B3 Accession No. V08/025855 together with at least one carrier.

In another embodiment, the at least one metabolite is an entomopathogenic agent, for example, the at least one metabolite is a secreted metabolite, such as a secreted toxin.

Preferably, said at least one carrier is an agriculturally acceptable carriers, more preferably is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant.

Preferably, said filler stimulant is a carbohydrate source, such as a disaccharide including, for example, sucrose, fructose, glucose, or dextrose, said anti-caking agent is selected from talc, silicon dioxide, calcium silicate, or kaelin clay, said wetting agent is skimmed milk powder, said emulsifier is a soy-based emulsifier such as lecithin or a vegetable-based emulsifier such as monodiglyceride, and said antioxidant is sodium glutamate or citric acid.

Preferably, said composition is a biological control composition, more preferably an entomopathogenic composition.

More preferably, said biological control composition is a stable composition capable of supporting reproductive viability of the fungi or capable of retaining entomopathogenic efficacy for a period greater than about two weeks, preferably greater than about one month, about two months, about three months, about four months, about five months, more preferably greater than about six months.

In certain embodiments, the composition comprises a single strain of fungi, Beauveria bassiana strain K4B3 (NMIA No. V08/025855 deposited 23 Sep. 2008).

Alternatively, the composition comprises multiple strains of said fungi, but preferably includes three strains or less. Suitably, the composition comprises NMIA No. V08/025855 together with any one or more strains selected from the group consisting of strain NMIA No. NM05/44593, strain NMIA No. NM05/44594, strain NMIA No. NM05/44595, strain NM06/00007, strain NM06/00008, strain NM06/00009, strain NM06/00010, and a strain having the identifying characteristics of any one of said strains.

Preferably, said composition is a biological control composition that comprises, in a reproductively viable form and amount, NMIA No. V08/025855 together with at least one strain selected from Lecanicillium muscarium strain K4V1 (NMIA No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof, together with at least one agriculturally acceptable carrier.

In other embodiments, the composition may additionally comprise at least one metabolite of B. bassiana strain K4B3 Accession No. V08/025855. For example, the composition is a biological control composition that comprises at least one metabolite of B. bassiana strain K4B3 Accession No. V08/025855 together with at least one strain selected from Lecanicillium muscarium strain K4V1 (NMIA No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof, together with at least one agriculturally acceptable carrier.

In still a further aspect, the invention provides a method of producing a composition comprising Beauveria bassiana V08/025855, optionally together with one or more other entomopathogenic fungi as described herein, said method comprising combining a reproductively viable form of said entomopathogenic fungi of the invention with at least one agriculturally acceptable diluent, carrier or excipient.

Preferably, said other fungi is selected from the group consisting of Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

In still a further aspect, the invention provides a method for producing a biological control composition, the method comprising:

providing a culture of Beauveria bassiana K4B3 V08/025855,

maintaining the culture under conditions suitable for production of at least one metabolite; and
i) combining the at least one metabolite with a carrier, or
ii) combining the at least one metabolite with one or more entomopathogenic fungi described herein, or
iii) separating the at least one metabolite from the Beauveria bassiana K4B3 V08/025855, or
iv) any combination of two or more of (i) to (iii).

In one embodiment, the metabolite is a secreted metabolite.

In another embodiment, the metabolite is an intracellular metabolite. Particularly in such embodiments, the method may additionally comprise after the maintaining step one or cell-lysis steps.

In various embodiments the separation is by centrifugation or by filtration.

In various embodiments, the separation is effective to remove greater than about 50% of the Beauveria bassiana K4B3 V08/025855, greater than about 55%, greater than about 60%, greater than about 65%, greater than about 70%, greater than about 75%, greater than about 80%, greater than about 85%, greater than about 90%, greater than about 95%, greater than about 99%, or about 100% of the Beauveria bassiana K4B3 V08/025855.

Accordingly, in one particularly contemplated embodiment, the method comprises providing a culture of Beauveria bassiana K4B3 V08/025855, maintaining the culture under conditions suitable for production of at least one secreted metabolite, and separating the at least one secreted metabolite from the Beauveria bassiana K4B3 V08/025855.

Preferably, the carrier is an agriculturally acceptable carrier, preferably the at least one carrier is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant.

The invention further provides the use of a composition of the invention for the control one or more phytopathogenic insects.

Preferably, said one or more phytopathogenic insects is selected from the group consisting of Thrips (Thysanoptera), Aphids, Psyllids, Scale or Whitefly (Hemiptera), caterpillars of Moths and Butterflies (Lepidoptera), and mites including Varroa mite.

In a further aspect the present invention provides a method for controlling one or more phytopathogenic insects, the method comprising applying to a plant or its surroundings a reproductively viable form and amount of Beauveria bassiana V08/025855, optionally together with at least one other entomopathogenic fungi as described herein.

In one embodiment, spores of the entomopathogenic fungi may be applied directly to the plant or its surroundings. Preferably, said spores are admixed with water and applied as described herein.

Preferably, said at least one other fungi is selected from the group consisting of Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

In a further aspect the present invention provides a method for controlling one or more phytopathogenic insects, the method comprising applying to a plant or its surroundings a composition of the present invention:

Preferably, the composition is admixed with water to a final concentration of about 0.5 gm/L to about 3 gm/L prior to application, and more preferably to a final concentration of about 1 gm/L.

Preferably, a desiccation protection agent, more preferably Fortune Plus™, is admixed to a final concentration of about 1 ml/L prior to application.

An exemplary concentration range is from about 1×10² to 1×10⁸ spores per ml, from about 1×10² to 1×10⁷ spores per ml, preferably from about 1×10³ to 2×10⁶, and more preferably 1×10⁴ to 2×10⁶ spores per ml.

Preferably, said composition comprises at least 10⁷ spores per milligram at application, more preferably, said application is by spraying.

Preferably, a composition comprising Beauveria bassiana strain K4B3 (NMIA Accession No. V08/025855) or a culture having the identifying characteristics thereof is applied at a rate of from about 1×10¹⁰ to about 1×10¹⁵ spores per hectare, preferably from about 1×10¹² to about 1×10¹⁴ spores per hectare, more preferably from about 5×10¹² to about 1×10¹⁴ spores per hectare, more preferably about 1-3×10¹³ spores per hectare.

Conveniently, such a rate of application can be achieved by formulating said composition at about 10⁷ spores per milligram or more, and applying said composition at a rate of about 1 kg per hectare. As discussed herein, such an application rate can be conveniently achieved by dissolution of the composition in a larger volume of agriculturally acceptable solvent, for example, water.

To those skilled in the art to which the invention relates, many changes in construction and differing embodiments and applications of the invention will suggest themselves without departing from the scope of the invention as defined in the appended claims. The disclosures and the descriptions herein are purely illustrative and are not intended to be in any sense limiting.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### DESCRIPTION OF FIGURES

FIG. 1 shows a mass spectrometry scan of the “beauvericin—normal methionine” standard as described in Example 4 herein. Peaks identified as beauvericin, beauvericin-F and bassianolide are shown.

FIG. 2 shows a mass spectrometry scan of an extract from the Beauveria bassiana K4B3 strain as described in Example 4 herein.

### DETAILED DESCRIPTION OF THE INVENTION

The present invention is in part directed to a strain of Beauveria bassiana having efficacy against phytopathogenic insects, and the use of such fungi in controlling said phytopathogenic insects.

### DEFINITIONS

The phrases “entomopathogenic activity” and “entomopathogenic efficacy” are used interchangeably herein and refer to the ability of certain agents, such as certain microorganisms, to antagonise one or more phytopathogenic insects.

Preferably, said entomopathogenic efficacy is the ability to parasitise and incapacitate, render infertile, impede the growth of, or kill one or more phytopathogenic insects, preferably within 14 days of contact with the insect, more preferably within 7 days, more preferably still the ability to kill one or more phytopathogenic insects within 7 days.

The term “biological control agent” (BCA) as used herein refers to a biological agent which acts as an antagonist of one or more phytopathogens, such as a phytopathogenic insect, or is able to control one or more phytopathogens. Antagonism may take a number of forms. In one form, the biological control agent may simply act as a repellent. In another form, the biological control agent may render the environment unfavourable for the phytopathogen. In a further, preferred form, the biological control agent may parasitise, incapacitate, render infertile, impeded the growth of, and/or kill the phytopathogen. Accordingly, the antagonistic mechanisms include but are not limited to antibiosis, parasitism, infertility, and toxicity. Therefore, agents which act as antagonists of one or more phytopathogenic insects can be said to have entomopathogenic efficacy. Furthermore, an agent that is an antagonist of a phytopathogenic insect can be said to be an entomopathogenic agent.

As used herein, a “biological control composition” is a composition comprising or including at least one biological control agent that is an antagonist of one or more phytopathogens. Such control agents include, but are not limited to, agents that act as repellents, agents that render the environment unfavourable for the pathogen, and agents that incapacitate, render infertile, and/or kill the pathogen.

Accordingly, as used herein an “entomopathogenic composition” is a composition which comprises or includes at least one agent that is an antagonist of one or more phytopathogenic insect. Such a composition is herein considered to have entomopathogenic efficacy.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “control” or “controlling” as used herein generally comprehends preventing, reducing, or eradicating phytopathogen infection or inhibiting the rate and extent of such infection, or reducing the phytopathogen population in or on a plant or its surroundings, wherein such prevention or reduction in the infection(s) or population(s) is statistically significant with respect to untreated infection(s) or population(s). Curative treatment is also contemplated. Preferably, such control is achieved by increased mortality amongst the phytopathogen population.

The term “metabolite” as used herein encompasses any substance produced by the fungi of the invention, or any substance taking part in a metabolic reaction occurring in the fungi of the invention, including any substance secreted, excreted or produced by the entomopathogenic fungi of the invention.

The term “plant” as used herein encompasses not only whole plants, but extends to plant parts, cuttings as well as plant products including roots, leaves, flowers, seeds, stems, callus tissue, nuts and fruit, bulbs, tubers, corms, grains, cuttings, root stock, or scions, and includes any plant material whether pre-planting, during growth, and at or post harvest. Plants that may benefit from the application of the present invention cover a broad range of agricultural and horticultural crops. The compositions of the present invention are also especially suitable for application in organic production systems.

When used in respect of an entomopathogenic agent, such as an entomopathogenic fungal strain, the phrase “retaining entomopathogenic efficacy” and grammatical equivalents and derivatives thereof is intended to mean that the agent still has useful entomopathogenic activity. Preferably, the retained activity is at least about 35, 40, 45, 50, 55, 60, 65, 70, 75, 80, 85, 90, 95, 99 or 100% of the original activity, and useful ranges may be selected between any of these values (for example, from about 35 to about 100%, from about 50 to about 100%, from about 60 to about 100%, from about 70 to about 100%, from about 80 to about 100%, and from about 90 to about 100%). For example, to be useful in the present invention a strain having the identifying characteristics of a specified strain should retain entomopathogenic activity, that is, retain at least about 35, 40, 45, 50, 55, 60, 65, 70, 75, 80, 85, 90, 95, 99 or 100% of the entomopathogenic activity of the specified strain. Accordingly, a strain having the identifying characteristics of B. bassiana K4B3, such as a homologue or mutant of B. bassiana K4B3, should retain at least about 35, 40, 45, 50, 55, 60, 65, 70, 75, 80, 85, 90, 95, 99 or 100% of the entomopathogenic activity of B. bassiana K4B3. Similarly, preferred compositions of the invention are capable of supporting the maintenance of useful entomopathogenic activity of the entomopathogenic agent (s) they comprise, and can be said to retain entomopathogenic activity, ideally until applied using the methods contemplated herein.

As used herein, the term “stable” when used in relation to a composition of the invention means a composition capable of supporting reproductive viability of the entomopathogenic fungi or of supporting entomopathogenic efficacy (for example of the one or more metabolites of the entomopathogenic fungi) for several weeks, preferably about one, about two, about three, about four, preferably about five, more preferably about six months, or longer.

A “strain having the identifying characteristics of [a specified strain]”, or a “culture having the identifying characteristics of [a specified culture]” including a homologue or mutant of the specified strain, is closely related to (i.e., shares a common ancestor with) or is derived from the specified strain, but will usually differ from the specified strain in one or more genotypic or phenotypic characteristics. Mutants are generally identifiable through assessment of genetic differences. Homologues are identifiable through assessment of the degree of genetic, biochemical and morphological difference and use of taxonomic methods, including for example analyses such as cladistics. However, a strain having the identifying characteristics of [a specified strain], including a homologue or mutant of the specified strain will retain entomopathogenic efficacy, will be distinguishable from other bacterial strains, and will be identifiable as a homologue or mutant of the parent strain using the techniques described herein.

The term “surroundings” when used in reference to a plant subject to the fungi, methods and compositions of the present invention includes soil, water, leaf litter, and/or growth media adjacent to or around the plant or the roots, tubers or the like thereof, adjacent plants, cuttings of said plant, supports, water to be administered to the plant, and coatings including seed coatings. It further includes storage, packaging or processing materials such as protective coatings, boxes and wrappers, and planting, maintenance or harvesting equipment.

Control of Phytopathogens

The present invention recognises that the horticultural sectors of many countries, including for example that of the United States of America, of New Zealand, and many states of Europe, are faced with the problem of increasing insecticide resistance amongst phytopathogenic insect pests. This is compounded under some regulatory regimes by a reduction in the availability of new chemical insecticides due to regulatory barriers.

The use of entomopathogenic fungi as biological control agents presents a solution to this problem. Effective biological control agents can be selected according their ability to incapacitate or kill a target phytopathogenic insect or insect population. Under conducive conditions, phytopathogenic insects such as aphids, thrips and whitefly may infect plants and their surroundings including soil, leaf litter, adjacent plants, supports, and the like. Entomopathogenic fungi may be applied so as to incapacitate and/or kill the phytopathogenic insect, thereby preventing or limiting the disease-causing capability of the pathogen. The effectiveness of these entomopathogenic fungi in the field is in turn dependent on their ability to survive varying climatic conditions, such as interrupted wet periods and desiccation.

The importation of entomopathogenic fungi is frequently problematic, costly, and impractical if not impossible under certain regulatory regimes. For example, entomopathogenic fungi available outside a given country may not be available to horticulturists within that country because of regulatory and legislative preclusions. The present invention therefore recognises there are distinct advantages to identifying and cultivating strains that are able to flourish under a wide variety of environmental conditions.

Isolates of said fungi may conveniently be obtained from the environment, including, for example, from plants, their surroundings, and from pathogens of said plants. In certain embodiments, isolates of said fungi may be obtained from the target insect, or from the plant species (or surroundings) to which the biological control agent comprising said fungi or a composition comprising said fungi will subsequently be applied.

Methods to determine growth of said fungi under different conditions, including different temperatures and on different media or other substrates, are well known in the art. Examples of methods to determine the ability of fungi to grow at various temperatures are described herein, as are methods to determine whether a given isolate is dead or dormant at a given temperature.

Similarly, methods to establish whether an isolate is able to grow on a given artificial medium are exemplified herein. The use of such methods recognises that an isolate must be capable of being grown in sufficient quantity for it to be suitable for use as a biological control agent. Methods of growing sufficient amounts of fungi of the invention are discussed further herein.

A strain of fungi, for example a strain of Beauveria, effective against phytopathogenic insects, and therefore suitable for use in accordance with the invention, is identified as one which is effective at reducing the population of the target insect species by a statistically significant amount with respect to the control treatment against which the strain or one or more metabolites of the strain is compared. Such strains can be considered as having entomopathogenic efficacy. As described herein, the reduction in the population of the target insect may be by various antagonistic mechanisms. For example, the fungi may parasitise, incapacitate, render infertile, and/or preferably kill the phytopathogenic insect. The fungi may also reduce the population of the target insect by rendering the environment, for example the plant to which the fungi is applied or its surroundings, unfavourable for the phytopathogenic insect. In this embodiment, the fungi may be considered to be acting as a repellent, and reducing the effective population of the target insect in the vicinity of the plant or its surroundings.

Preferably, suitable strains exhibit about 5% entomopathogenic efficacy, at least about 10%, at least about 15%, at least about 20%, at least about 25%, at least about 30%, at least about 35%, at least about 40%, at least about 45%, more preferably at least about 50% entomopathogenic efficacy expressed as a percentage reduction of the population of the relevant insect species compared to the control treatment. By way of illustration, the methodology described herein was employed to identify a Beauveria isolate effective against a variety of target insects, whereas procedures analogous to those described herein can be employed in relation to other fungi and insect species.

Although entomopathogenic efficacy is a principal requisite for an isolate to be considered suitable for use as a biological control agent, the fungal isolate should have additional characteristics to be suitable for use as a biological control agent.

For example, the fungi should be able to be stored in a viable form for a reasonable period, ultimately so as to allow it to be applied to the target plant or its surroundings in a form and concentration that is effective as a biological control agent.

The fungi should also be able to achieve infection threshold when applied to a plant or its surroundings for it to be suitable for use as a biological control agent. As used herein, infection threshold refers to the concentration of fungi required for the fungi to become established on the target plant or its surroundings so as to then have entomopathogenic efficacy. As will be appreciated, in order to achieve infection threshold, some isolates of fungi may require application at such a high rate as to be impractical or unviable. Furthermore, some fungal isolates may not be able to achieve infection threshold irrespective of the concentration or rate at which they are applied. Suitable entomopathogenic fungi are able to achieve infection threshold when applied at a rate of not less that 10¹⁰ spores per hectare, or applied at a concentration not less than 10⁷ spores per milligram of composition when said composition is applied at a rate of about 1 kg/1000 L/hectare.

Methods to determine infection threshold are well known in the art, and examples of such methods are presented herein. In certain embodiments, infection threshold can be determined directly, for example by analysing one or more samples obtained from a target plant, its surroundings, and/or a pathogen of said plant, and determining the presence or amount of fungus on or in said sample. In other embodiments, infection threshold can be determined indirectly, for example by observing a reduction in the population of one or more phytopathogenic insects. Combinations of such methods are also envisaged.

Beauveria bassiana is a soil born fungi that attacks both immature and adult insects including, for example, grasshoppers, aphids, thrips, moths, and several other species. Typically, B. bassiana can be isolated from insect cadavers, such as aphids, borers, and thrips, and may also be isolated from soil. The entomopathogenic Beauveria bassiana strain K4B3 of the invention is described in more detail below.

Mycelium: Grows readily on MEA. Colonies are generally white at the edge becoming cream to pale yellow. Very occasionally reddish. Underside of mycelium thallus infuses a red blush pigment into agar.
Conidiophores: Abundant, rising from hyphae. 1-2 μm wide bearing groups of clustered conidiogenous cells 3-6×3-5 μm which may branch to give rise to further conidiogenous cells, globular to flask shape with well developed stalk up to 20 μm long by 1 μm wide, geniculate with denticles up to 1 μm wide.
Conidia: Clear globose conidia that are 2-3×2-2.5 μm. Blastospores are formed in submerged culture. Hydrophobic. Dusty, granular appearance in aggregation on agar. K4B3 on agar produces very clumped granular aggregations. The colour of the spores aggregations changes to a deep almost iridescent yellow in colour at maturity. Introduction of K4B3 into submerged culture produces an extreme red colour and an acrid metallic odor.

Beauveria bassiana strain K4B3 was isolated from a group of dead cicada pupae. Details of the isolation and selection process employed to obtain this isolate are set out in the Examples. This B. bassiana isolate has been deposited in the National Measurement Institute of Australia (NMIA, formerly the Australian Government Analytical Laboratories (AGAL)), 1 Suakin Street, Pymble, New South Wales, Australia on 23 Sep. 2008 according to the Budapest Treaty for the purposes of patent procedure. The isolate has been accorded the deposit number V08/025855.

Accordingly, in one aspect the present invention provides a biologically pure culture of B. bassiana strain K4B3, NMIA No. V08/025855. Similarly provided are Beauveria having the identifying characteristics of strain K4B3, NMIA No. V08/025855.

B. bassiana strain K4B3 is a particularly effective biological control agent, being capable of surviving interrupted wet periods, desiccation, and colonising, incapacitating and killing phytopathogenic insects such as, but not limited to, aphids, caterpillars, whitefly, moths, Varroa mite, cicada, and thrips in the field. The degree of killing of whitefly, thrips, and aphids using a blastospore or condial composition by this isolate of Beauveria bassiana is generally as good as the commonly used insecticides employed by growers. However, resistance to such insectides by insects, for example thrips, whitefly and aphids, has become the greatest threat to the horticultural industry.

For example, overseas populations of Western flower thrips are resistant to most groups of pesticides. The following pesticides gave inadequate control in the 24 hour bioassay: acephate, abamectin, chlorpyrifos, endosulfan, methomyl, methiocarb, omethoate, pyrazophos, and tau-fluvalinate.

In New Zealand, resistance to deltamethrin is present in Onion thrips in the North and South Islands, but resistance to diazinon and dichlorvos has only been found near Auckland (Martin et al. in prep). Onion thrips have been reported to be resistant to many pesticides in the USA, but still susceptible to synthetic pyrethroids (Grossman 1994).

Resistance to chlorpyrifos in Kelly's citrus thrips has been reported from South Australia (Purvis 2002).

There have been reports of insecticide resistance to greenhouse whitefly, but only the most recent, to buprofezin, has been confirmed (Workman & Martin 1995). Overseas, greenhouse whitefly has developed resistance to organochlorine, organophosphate, carbamate and pyrethroid insecticides (e.g. Georghiou 1981, Anis & Brennan 1982, Elhag & Horn 1983, Wardlow 1985, and Hommes 1986). Resistance has also been found in newer insecticides, buprofezin and teflubenzuron (Gorman et al. 2000).

It is therefore apparent that many plant pathogenic insects have developed resistance to a number of insecticides; in these and other instances, Beauveria bassiana isolates selected in accordance with the invention provide an effective alternative for insect control. This potent activity in the control of plant disease coupled with the absence of any observations of plant pathogenicity induced by Beauveria bassiana K4B3 or one or more metabolites thereof demonstrate this isolate has desirable attributes for use as a biological control agent.

In other embodiments of the present invention, B. bassiana K4B3 may be used to prepare a composition comprising one or more metabolites of B. bassiana K4B3, wherein the one or more metabolite is an entomopathogenic agent.

As described above, when grown under conducive conditions the mycelium of B. bassiana K4B3 is reddish, and when grown on agar B. bassiana K4B3 infuses a red pigment into the agar. Similarly, as described herein in Example 1, when grown in submerged culture B. bassiana K4B3 produces an extreme red colour and an acrid metallic odour and infuses one or more toxic metabolites into the culture solution. Compositions comprising one or more such toxic metabolites are specifically contemplated herein. One exemplary composition comprises the media in which B. bassiana K4B3 has been grown or maintained, whether or not B. bassiana K4B3 has subsequently been removed from the media. A further exemplary composition is media in which B. bassiana K4B3 has been grown or maintained or an extract of media in which B. bassiana K4B3 has been grown or maintained having a mass spectrometric profile characteristic of that depicted herein in FIG. 2.

Accordingly, the invention provides methods for producing a composition comprising one or more metabolites of B. bassiana K4B3, and particularly one or more secreted metabolites of B. bassiana K4B3.

In one embodiment, the method comprises maintaining a culture of Beauveria bassiana K4B3 V08/025855 under conditions suitable for production of at least one metabolite; and separating the at least one metabolite from the Beauveria bassiana K4B3 V08/025855.

In one embodiment, the composition comprises one or more of beauvericin, beauvericin-F, and bassianolide, preferably two or more of beauvericin, beauvericin-F, and bassianolide. In one embodiment, the composition is a synergistic composition comprising beauvericin, beauvericin-F, and bassianolide.

In another embodiment, the composition comprises less than about 1 mgL⁻¹ beauvericin, less than about 0.5 mgL⁻¹ beauvericin, less than about 0.1 mgL⁻¹ beauvericin, less than about 0.05 mgL⁻¹ beauvericin, less than about 0.01 mgL⁻¹ beauvericin, less than about 0.005 mgL⁻¹ beauvericin, less than about 0.001 mgL⁻¹ beauvericin, less than about 0.0005 mgL⁻¹ beauvericin, or less than about 0.0001 mgL⁻¹ beauvericin.

In another embodiment, the composition comprises less than about 1 mgL⁻¹ beauvericin-F, less than about 0.5 mgL⁻¹ beauvericin-F, less than about 0.1 mgL⁻¹ beauvericin-F, less than about 0.05 mgL⁻¹ beauvericin-F, less than about 0.01 mgL⁻¹ beauvericin-F, less than about 0.005 mgL⁻¹ beauvericin-F, less than about 0.001 mgL⁻¹ beauvericin-F, less than about 0.0005 mgL⁻¹ beauvericin-F, or less than about 0.0001 mgL⁻¹ beauvericin-F.

Beauveria bassiana strain K4B3 of the invention may be used singly, or in combination with other entomopathogenic fungi described herein. Examples of other entomopathogenic fungi are described in more detail below.

Beauveria bassiana strain K4B1 was isolated from a borer larva within a pine forest in Bombay, New Zealand. This B. bassiana isolate has been deposited in the National Measurement Institute of Australia, 1 Suakin Street, Pymble, New South Wales, Australia on 16 Mar. 2005 according to the Budapest Treaty for the purposes of patent procedure. The isolate has been accorded the deposit number NM05/44595.

Beauveria bassiana isolate K4B1 shows a preference for thrips adults, and is also pathogenic to thrip juveniles and pupae, aphids and whitefly. The conidia of K4B1 form cream aggregations.

Beauveria bassiana isolate K4B2 was isolated from a Lepidoptera caterpillar on a sunflower in the Aka Aka flats, New Zealand. This B. bassiana isolate has been deposited in the National Measurement Institute of Australia on 3 Mar. 2006 according to the Budapest Treaty for the purposes of patent procedure. The isolate has been accorded the deposit number NM06/00010.

Beauveria bassiana isolate K4B2 exhibits a preference for caterpillars, including soybean looper caterpillar and white butterfly and army worm caterpillar. This isolate is also pathogenic to thrip juveniles, adults, and pupae, aphids and whitefly. The conidia of K4B2 form yellow dusty aggregations.

NMIA No. NM05/44595, NMIA No. NM06/00010 and other suitable isolates of B. bassiana may be used in combination with the Beauveria bassiana strain K4B3 of the invention, or in combination with one or more metabolites of B. bassiana K4B3, and are particularly effective biological control agents, being capable of surviving interrupted wet periods, desiccation, and colonising, incapacitating and killing phytopathogenic insects such as, but not limited to, aphids, caterpillars, whitefly, moths, Varroa mite and thrips in the field. The degree of killing of whitefly, thrips and aphids by these isolates of B. bassiana is generally as good as the commonly used insecticides as described above. Resistance to these insecticides has developed; in these and other instances, B. bassiana isolates selected in accordance with the invention provide an effective alternative for insect control. This potent activity in the control of plant disease coupled with the absence of any observations of plant pathogenicity induced by B. bassiana demonstrate that isolates of these species have desirable attributes for use as a biological control agent.

Lecanicillium muscarium is an entomopathogenic fungi with a broad host range including homopteran insects and other arthropod groups. L. muscarium is considered a species complex, which includes isolates of varied morphological and biochemical characteristics. Typically, L. muscarium can be isolated from insect cadavers, such as aphids, thrips, whitefly, and mealy bugs, and may also be isolated from soil.

Isolates have the following identifying characteristics:

Mycelium: Colonies on potato dextrose agar (PDA), malt extract agar (MEA) or oatmeal agar (OA) are white, creamy, thin, cottony, with reverse colourless to pale or deep yellow.
Conidiophores: Phialides are formed singly or directly from mycelium or in whorls of 3 or 4 erect conidiophores much like vegetative mycelium. Phialides delicate, of variable size depending on both strain and the age of the culture. Size ranges from 8.5-16×0.8-1.2 μm to 30-40×2-2.2 μm.
Conidia: Produced singly and aggregating on heads at the tips of the phialides in a mucilaginous matrix. Ellipsoidal to cylindrical with rounded ends varying in size with the strain from 2.3-3.4×1-1.3 μm to 7.2-10×2.1-2.6 m. Blastospores are produced in submerged culture. Hydrophilic.

Lecanicillium muscarium strain K4V1 was isolated from whitefly in a greenhouse tomato crop in Pukekohe, New Zealand. This L. muscarium isolate has been deposited in the National Measurement Institute of Australia on 16 Mar. 2005 according to the Budapest Treaty for the purposes of patent procedure. The isolate has been accorded the deposit number NM05/44593.

K4V1 has the additional identifying characteristics—60% Conidia 1.0×1.0 micron on whitefly scale, 30% Conidia 2.0×1.0 micron on thrip juveniles (nymphs), 10% Conidia 2.5×1.3 micron on thrip pupae. Underside of mycelium thallus sparsely creased, Mycelium thallus removes from the agar very easily.

L. muscarium strain K4V2 was isolated from whitefly in a cucumber greenhouse in Ruakaka, New Zealand. This L. muscarium isolate has been deposited in the National Measurement Institute of Australia on 16 Mar. 2005 according to the Budapest Treaty for the purposes of patent procedure. The isolate has been accorded the deposit number NM05/44594.

K4V2 has the additional identifying characteristics—50% Conidia 2.0×1.5 μm, 30% Conidia 2.0×1.0 μm, 20% Conidia 1.0×1.0 μm, pathogenic to Whitefly adults, while Blastospores pathogenic to aphids. Underside of mycelium thallus frequently creased, Mycelium thallus difficult to remove from agar surface.

L. muscarium strain K4V4 was isolated from isolated from an outdoor organic tamarillo crop. This L. muscarium isolate has been deposited in the National Measurement Institute of Australia on 3 Mar. 2006 according to the Budapest Treaty for the purposes of patent procedure. The isolate has been accorded the deposit number NM06/00007.

K4V4 has the additional identifying characteristics—50% Conidia 1.0×0.5 μm, pathogenic to whitefly scale and adults, very aggressive at low humidity 65-75%, high temp 28-32°. Generally v.1 >75%. 50% Condidia 0.5×0.5 μm. Underside of mycelium thallus sparsely creased, Mycelium thallus diffuses custard yellow to light orange pigment in media.

NMIA No. NM05/44593, NMIA No. NM05/44594, NMIA No. NM06/00007 and other suitable isolates of L. muscarium may be used in combination with the Beauveria bassiana K4B3 strain of the invention, or in combination with one or more metabolites of B. bassiana K4B3, and are particularly effective biological control agents, being capable of surviving interrupted wet periods, desiccation, and colonising, incapacitating and killing phytopathogenic insects such as, but not limited to, aphids, whitefly, mealy bugs, Varroa mite, and thrips, in the field. The degree of killing of whitefly, thrips and aphids by these isolates of L. muscarium is generally as good as the commonly used insecticides as described above. Resistance to these insecticides has developed; in these and other instances, L. muscarium isolates selected in accordance with the invention provide an effective alternative for insect control. This potent activity in the control of plant disease coupled with the absence of any observations of plant pathogenicity induced by L. muscarium demonstrate that isolates of these species have desirable attributes for use as a biological control agent.

Lecanicillium longisporum is an entomopathogenic fungi that is particularly pathogenic to aphids. Lecanicillium longisporum strain KT4L1 was isolated from aphids in Barley grass Banker plants in Franklin, Auckland, New Zealand. This L. longisporum isolate has been deposited in the National Measurement Institute of Australia on 3 Mar. 2006 according to the Budapest Treaty for the purposes of patent procedure. The isolate has been accorded the deposit number NM06/00009.

The isolate KT4L1 has the following identifying characteristics: 100% Condidia 6.0×2.1 μm, Mycelium thallus is offwhite to yellow growing very roughly which could be described as lumpy in consistency. Mycelium thallus diffuses light red brown colour into agar.

NMIA No. NM06/00009 and other suitable isolates of L. longisporum may be used in combination with the Beauveria bassiana K4B3 strain of the invention, or in combination with one or more metabolites of B. bassiana K4B3, and are particularly effective biological control agents, being capable of surviving interrupted wet periods, desiccation, and colonising, incapacitating and killing phytopathogenic insects such as aphids, in the field. The degree of killing of aphids using a blastospore or condial composition by these isolates of L. longisporum is generally as good as the commonly used insecticides employed by growers.

As discussed above, many plant pathogenic insects have developed resistance to a number of insecticides; in these and other instances, L. longisporum isolates selected in accordance with the invention provide an effective alternative for insect control. This potent activity in the control of plant disease coupled with the absence of any observations of plant pathogenicity induced by L. longisporum demonstrate that isolates of these species have desirable attributes for use as a biological control agent.

Paecilomyces fumosoroseus is an entomopathogenic fungi found in infected and dead insects, and in some soils. P. fumosoroseus typically infects whiteflies, thrips, aphids, and caterpillars.

The K4P1 strain of Paecilomyces fumosoroseus meeting the above requirements was isolated from Diamond Back Moth caterpillar present on cabbage in Runciman, New Zealand. This P. fumosoroseus isolate has been deposited in the National Measurement Institute of Australia on 3 Mar. 2006 according to the Budapest Treaty for the purposes of patent procedure. The isolate has been accorded the deposit number NM06/00008.

P. fumosoroseus strain K4P1 has the following identifying characteristics:

Growth on insect: Produces simple mononematous conidiophores or distinct but loose synnemata. The synnemata are erect, up to 3 cm long and maybe branched, appearing dusty with conidia.

Growth on agar: On malt agar (MA) and PDA, growth is moderately rapid at room temperature (25° C.) 4-8 cm in 14 days, with a basal felt with regular or irregular raised floccose overgrowth, or maybe thinner, dusty and granular, and producing definite coremia which are powdery when first isolated. White at first, remaining so or changing to shades of pink which may become tinged grey with age.

Vegetative hyphae: Smooth walled, hyaline, 1-5-3.5 μm diameter.
Conidial structures: Tend to be complex consisting of erect conidiophores arising from the basal felt or from aerial hyphae.
Conidiophores: Produced singly or together to form synnemata, up to 100 μm long×1.5-2 (3) μm diameter. Smooth walled, hyaline, bearing verticils of branches, in turn bearing whorls of 3-6 phialides, occasional phialides produced at the same level as the branches and in the same verticil. Sometimes the verticillate pattern is broken and single branches are produced irregularly on the conidiophore.
Phialides: 5-7×2.5 (3) μm, with a swollen base which tapers to a long thin neck about 0.5 μm diameter.
Conidia: Cylindrical to fusiform with rounded ends, smooth, hyaline, borne in chains, 2-4×1-2 μm, occasionally up to 5 μm long.

On insects the conidiogenous apparatus tends to be more compacted with the branches and phialides inflated, slightly shorter and more rounded, 3.5-6×1-2.5 μm. Conidia as in culture.

NMIA No. NM06/00008 and other suitable isolates of P. fumosoroseus may be used in combination with the Beauveria bassiana K4B3 strain of the invention, or in combination with one or more metabolites of B. bassiana K4B3, and are particularly effective biological control agents, being capable of surviving interrupted wet periods, desiccation, and colonising, incapacitating and killing phytopathogenic insects such as, but not limited to, whitefly, Varroa mite, and Lepidoptera caterpillar in the field. The degree of killing of whitefly, Varroa mite, and thrips, and aphids using a blastospore or condial composition by these isolates of P. fumosoroseus is generally as good as the commonly used insecticides employed by growers.

As discussed above, many plant pathogenic insects have developed resistance to a number of insecticides; in these and other instances, P. fumosoroseus isolates selected in accordance with the invention provide an effective alternative for insect control. This potent activity in the control of plant disease coupled with the absence of any observations of plant pathogenicity induced by P. fumosoroseus demonstrate that isolates of these species have desirable attributes for use as a biological control agent.

In a further aspect the present invention provides a composition which comprises B. bassiana strain K4B3, or one or more metabolites of B. bassiana K4B3, or comprises both B. bassiana K4B3 and one or more metabolites of B. bassiana K4B3, optionally with one or more other entomopathogenic fungi, together with at least one carrier.

The composition may include multiple strains of entomopathogenic fungi, and in certain embodiments, multiple strains may be utilised to target a number of phytopathogenic species, or a number of different developmental stages of a single phytopathogen, or indeed a combination of same. For example, the pupal form of a phytopathogenic insect may be targeted with one fungal strain, while the adult form of the phytopathogenic insect may be targeted with another fungal strain, wherein both strains are included in a composition of the invention. In other embodiments, three strains or less will be preferred, and frequently a single strain will be preferred.

Suitably, the composition comprises fungi selected from the group consisting of Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

Particularly contemplated are compositions comprising one or more metabolites of B. bassiana K4B3 and Lecanicillium muscarium strain K4V1 (NM05/44593) or a strain having the identifying characteristics thereof, compositions comprising one or more metabolites of B. bassiana K4B3 and Lecanicillium muscarium strain K4V2 (NM05/44594) or a strain having the identifying characteristics thereof, and compositions comprising one or more metabolites of B. bassiana K4B3 and both Lecanicillium muscarium strain K4V1 (NM05/44593) or a strain having the identifying characteristics thereof, compositions comprising one or more metabolites of B. bassiana K4B3 and Lecanicillium muscarium strain K4V2 (NM05/44594) or a strain having the identifying characteristics thereof.

Examples of compositions comprising entomopathogenic fungi are well known in the art, and include those described in, for example, WO95/10597 (published as PCT/US94/11542) to Mycotech Corporation, WO2003/043417 (published as PCT/US2002/037218) to The United States of America as represented by The Secretary of Agriculture, U.S. Pat. No. 4,530,834 to McCabe et al., and U.S. patent application Ser. No. 10/657,982 (published as US 2004/0047841) to Wright et al., each incorporated by reference herein in its entirety.

To be suitable for application to a plant or its surroundings, said at least one carrier is an agriculturally acceptable carrier, more preferably is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant. Preferably, said filler stimulant is a carbohydrate source, such as a disaccharide including, for example, sucrose, fructose, glucose, or dextrose, said anti-caking agent is selected from talc, silicon dioxide, calcium silicate, or kaelin clay, said wetting agent is skimmed milk powder, said emulsifier is a soy-based emulsifier such as lecithin or a vegetable-based emulsifier such as monodiglyceride, and said antioxidant is sodium glutamate or citric acid. However, other examples well known in the art may be substituted, provided the ability of the composition to support fungal viability is maintained.

Preferably, said composition is a biological control composition. The concentration of the entomopathogenic fungi of the invention or the one or more metabolites thereof present in the composition that is required to be effective as biological control agents may vary depending on the end use, physiological condition of the plant; type (including insect species), concentration and degree of pathogen infection; temperature, season, humidity, stage in the growing season and the age of plant; number and type of conventional insecticides or other treatments (including fungicides) being applied; and plant treatments (such as deleafing and pruning) may all be taken into account in formulating the composition.

For use as a biological control agent, when present in the composition the entomopathogenic fungi of the invention should be in a reproductively viable form. The term reproductively viable as used herein includes mycelial and spore forms of the fungi. For example, for most purposes, fungal strains are desirably incorporated into the composition in the form of spores (conidia or blastospores). Spores are obtainable from all the fungal strains of the invention, and may be produced using known art techniques. Spores obtained from the fungal strains of the invention form a further aspect of the invention. The concentration of the fungal spores in the composition will depend on the utility to which the composition is to be put. An exemplary concentration range is from about 1×10⁶ to 1×10¹² spores per ml, preferably from about 1×10⁷ to 2×10¹⁰, and more preferably 1×10⁷ to 1×10⁸ spores per ml.

In theory one infective unit should be sufficient to infect a host but in actual situations a minimum number of infective units are required to initiate an infection. The concept of lethal dose (LD) regularly used with chemical pesticides is inappropriate for microbial pesticides in which entomopathogenic efficacy is reliant on colonisation of the plant or its surroundings by the entomopathogenic fungi. Concepts of infective dose (ID) or infective concentration (IC) are more precise or applicable. ID or IC refer to the actual number of infective units needed to initiate infection or the number of infective units exposed to the pathogen to cause death. Therefore, the number of infective units applied in the field or greenhouse against a pathogen will affect the degree of control. It is important to apply the desired concentration of the anti-phytopathogenic bacteria, property placed and at the right time, to obtain good control of the pest: this is known as the “infection threshold”.

It will be apparent that the concentration of fungal spores in a composition formulated for application may be less than that in a composition formulated for, for example, storage. The Applicants have determined that with the entomopathogenic fungi of the present invention, infection threshold occurs at about 10⁷ spores per ml of sprayable solution, when applied at a rate of about 1 L per hectare. Accordingly, in one example, a composition formulated for application will preferably have a concentration of at least about 10⁷ spores per ml. In another example, a composition formulated for storage (for example, a composition such as a wettable powder capable of formulation into a composition suitable for application) will preferably have a concentration of about 10¹⁰ spores per gram. It will be apparent that the spore concentration of a composition formulated for storage and subsequent formulation into a composition suitable for application must be adequate to allow said composition for application to also be sufficiently concentrated so as to be able to be applied to reach infection threshold.

Preferably, the composition is a stable composition capable of supporting reproductive viability of the fungi or entomopathogenic efficacy (for example of one or more metabolites) for a period greater than about two weeks, preferably greater than about one month, about two months, about three months, about four months, about five months, more preferably greater than about six months. To be suitable for use as a biological control composition, the composition preferably is able to support reproductive viability of the fungi or entomopathogenic efficacy for a period greater than about six months.

Using conventional solid substrate and liquid fermentation technologies well known in the art, the entomopathogenic fungi of the invention can be grown in sufficient amounts to allow use as biological control agents. For example, spores from selected strains can be produced in bulk for field application using nutrient film, submerged culture, and rice substrate growing techniques. Similarly, metabolites of the fungi of the invention may be produced in sufficient quantity using these growing techniques, and exemplary techniques are presented herein in the Examples. Growth is generally effected under aerobic conditions at any temperature satisfactory for growth of the organism. For example, for B. bassiana, a temperature range of from 10 to 32° C., preferably 25 to 30° C., and most preferably 23° C., is preferred. The pH of the growth medium is slightly acid to neutral, that is, about 5.0 to 7.0, and most preferably 5.5. Incubation time is sufficient for the isolate to reach a stationary growth phase, about 21 days when incubated at 23° C., and will occur in normal photoperiod.

The spores may be harvested by methods well known in the art, for example, by conventional filtering or sedimentary methodologies (eg. centrifugation) or harvested dry using a cyclone system. Spores can be used immediately or stored, chilled at 0° to 6° C., preferably 2° C., for as long as they remain reproductively viable. It is however generally preferred that when not incorporated into a composition of the invention, use occurs within two weeks of harvesting.

Similarly, when required, the one or more metabolites of B. bassiana K4B3 may be separated from the B. bassiana K4B3 by methods well known in the art, for example, by conventional filtering or sedimentary methodologies (eg. centrifugation), whether in combination with one or more cell-lysis steps (for example, for intracellular metabolites) or not (for example, for metabolites that are secreted into the growth media).

The composition of the invention may also include one or more carriers, preferably one or more agriculturally acceptable carriers. In one embodiment the carrier, such as an agriculturally acceptable carrier, can be solid or liquid. Carriers useful herein include any substance typically used to formulate agricultural composition.

In one embodiment the agriculturally acceptable carrier maybe selected from the group comprising fillers, solvents, excipients, surfactants, suspending agents, speaders/stickers (adhesives), antifoaming agents, dispersants, wetting agents, drift reducing agents, auxiliaries, adjuvants or a mixture thereof.

Compositions of the invention may be formulated as, for example, concentrates, solutions, sprays, aerosols, immersion baths, dips, emulsions, wettable powders, soluble powders, suspension concentrates, dusts, granules, water dispersible granules, microcapsules, pastes, gels and other formulation types by well-established procedures.

These procedures include mixing and/or milling of the active ingredients with agriculturally acceptable carrier substances, such as fillers, solvents, excipients, surfactants, suspending agents, speaders/stickers (adhesives), antifoaming agents, dispersants, wetting agents, drift reducing agents, auxiliaries and adjuvants.

In one embodiment solid carriers include but are not limited to mineral earths such as silicic acids, silica gels, silicates, talc, kaolin, attapulgus clay, limestone, lime, chalk, bole, loess, clay, dolomite, diatomaceous earth, aluminas calcium sulfate, magnesium sulfate, magnesium oxide, ground plastics, fertilizers such as ammonium sulfate, ammonium phosphate, ammonium nitrate, and ureas, and vegetable products such as grain meals, bark meal, wood meal, and nutshell meal, cellulosic powders and the like. As solid carriers for granules the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

In one embodiment the carrier may also be liquid, for example, water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulfoxide, or N-methylpyrrolidone; liquefied gases; or the like or a mixture thereof.

In one embodiment surfactants include nonionic surfactants, anionic surfactants, cationic surfactants and/or amphoteric surfactants and promote the ability of aggregates to remain in solution during spraying.

Spreaders/stickers promote the ability of the compositions of the invention to adhere to plant surfaces. Examples of surfactants, spreaders/stickers include but are not limited to Tween and Triton (Rhom and Hass Company), Fortune®, Pulse, C. Daxoil®, Codacide oil®, D-C. Tate®, Supamet Oil, Bond®, Penetrant, Glowelt® and Freeway, Citowett®, Fortune Plus™, Fortune Plus Lite, Fruimec, Fruimec lite, alkali metal, alkaline earth metal and ammonium salts of aromatic sulfonic acids, e.g., ligninsulfonic acid, phenolsulfonic acid, naphthalenesulfonic acid and dibutylnaphthalenesulfonic acid, and of fatty acids, alkyl and alkylaryl sulfonates, and alkyl, lauryl ether and fatty alcohol sulfates, and salts of sulfated hexadecanols, heptadecanols, and octadecanols, salts of fatty alcohol glycol ethers, condensation products of sulfonated naphthalene and naphthalene derivatives with formaldehyde, condensation products of naphthalene or naphthalenesulfonic acids with phenol and formaldehyde, polyoxyethylene octylphenol ethers, ethoxylated isooctylphenol, ethoxylated octylphenol and ethoxylated nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methyl cellulose. Where selected for inclusion, one or more agricultural surfactants, such as Tween are desirably included in the composition according to known protocols.

Wetting agents reduce surface tension of water in the composition and thus increase the surface area over which a given amount of the composition may be applied. Examples of wetting agents include but are not limited to salts of polyacrylic acids, salts of lignosulfonic acids, salts of phenolsulfonic or naphthalenesulfonic acids, polycondensates of ethylene oxide with fatty alcohols or fatty acids or fatty esters or fatty amines, substituted phenols (particularly alkylphenols or arylphenols), salts of sulfosuccinic acid esters, taurine derivatives (particularly alkyltaurates), phosphoric esters of alcohols or of polycondensates of ethylene oxide with phenols, esters of fatty acids with polyols, or sulfate, sulfonate or phosphate functional derivatives of the above compounds.

In one embodiment the preferred method of applying the compound or composition of the invention is to spray a dilute or concentrated solution by handgun or commercial airblast.

As described above, the compositions of the present invention may be used alone or in combination with one or more other agricultural agents, including pesticides, insecticides, acaracides, fungicides (provided such fungicides are not detrimental or toxic to the fungi of the invention), bactericides, herbicides, antibiotics, antimicrobials, nemacides, rodenticides, entomopathogens, pheromones, attractants, plant growth regulators, plant hormones, insect growth regulators, chemosterilants, microbial pest control agents, repellents, viruses, phagostimulents, plant nutrients, plant fertilisers and biological controls. When used in combination with other agricultural agents the administration of the two agents may be separate, simultaneous or sequential. Specific examples of these agricultural agents are known to those skilled in the art, and many are readily commercially available.

Examples of plant nutrients include but are not limited to nitrogen, magnesium, calcium, boron, potassium, copper, iron, phosphorus, manganese, molybdenum, cobalt, boron, copper, silicon, selenium, nickel, aluminum, chromium and zinc.

Examples of antibiotics include but are not limited to oxytetracyline and streptomycin.

Examples of fungicides include but are not limited to the following classes of fungicides: carboxamides, benzimidazoles, triazoles, hydroxypyridines, dicarboxamides, phenylamides, thiadiazoles, carbamates, cyano-oximes, cinnamic acid derivatives, morpholines, imidazoles, beta-methoxy acrylates and pyridines/pyrimidines.

Further examples of fungicides include but are not limited to natural fungicides, organic fungicides, sulphur-based fungicides, copper/calcium fungicides and elicitors of plant host defences.

Examples of natural fungicides include but are not limited to whole milk, whey, fatty acids or esterified fatty acids.

Examples of organic fungicides include but are not limited to any fungicide which passes an organic certification standard such as biocontrol agents, natural products, elicitors (some of may also be classed as natural products), and sulphur and copper fungicides (limited to restricted use).

An example of a sulphur-based fungicide is Kumulus™ DF (BASF, Germany).

An example of a copper fungicide is Kocide® 2000 DF (Griffin Corporation, USA).

Examples of elicitors include but are not limited to chitosan, Bion™, BABA (DL-3-amino-n-butanoic acid, β-aminobutyric acid) and Milsana™ (Western Farm Service, Inc., USA).

In some embodiments non-organic fungicides may be employed. Examples of non-organic fungicides include but are not limited to Bravo™ (for control of PM on cucurbits); Supershield™ (Yates, NZ) (for control of Botrytis and PM on roses); Topas® 200EW (for control of PM on grapes and cucurbits); Flint™ (for control of PM on apples and cucurbits); Amistar® WG (for control of rust and PM on cereals); and Captan™, Dithane™, Euparen™, Rovral™, Scala™, Shirlan™, Switch™ and Teldor™ (for control of Botrytis on grapes).

Examples of pesticides include but are not limited to azoxystrobin, bitertanol, carboxin, Cu₂O, cymoxanil, cyproconazole, cyprodinil, dichlofluamid, difenoconazole, diniconazole, epoxiconazole, fenpiclonil, fludioxonil, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, metconazole, oxadixyl, pefurazoate, penconazole, pencycuron, prochloraz, propiconazole, pyroquilone, SSF-109, spiroxamin, tebuconazole, thiabendazole, tolifluamid, triazoxide, triadimefon, triadimenol, triflumizole, triticonazole and uniconazole.

An example of a biological control agent other than a fungal strain of the present invention is the BotryZen™ biological control agent comprising Ulocladium oudemansii.

The compositions may also comprise a broad range of additives such as stablisers and penetrants used to enhance the active ingredients and so-called ‘stressing’ additives to improve spore vigor, germination and survivability such as potassium chloride, glycerol, sodium chloride and glucose. Additives may also include compositions which assist in maintaining microorganism viability in long term storage, for example unrefined corn oil and so called invert emulsions containing a mixture of oils and waxes on the outside and water, sodium alginate and conidia on the inside.

It is important that any additives used are present in amounts that do not interfere with the effectiveness of the biological control agents.

Examples of suitable compositions including carriers, preservations, surfactants and wetting agents, spreaders, and nutrients are provided in U.S. Pat. No. 5,780,023, incorporated herein in its entirety by reference.

The Applicants have also determined that many commonly used fungicides do not adversely affect the entomopathogenic fungi of the invention. The compositions of the invention may therefore also include such fungicides. Alternatively, the compositions may be used separately but in conjunction with such fungicides in control programmes.

The invention also provides a method of producing a composition comprising one or more entomopathogenic fungi of the invention, said method comprising obtaining a reproductively viable form of said entomopathogenic fungi, and combining said reproductively viable form of said entomopathogenic fungi with at least one agriculturally acceptable carrier.

The compositions may be prepared in a number of forms. One preparation comprises a powdered form of a composition of the invention which may be dusted on to a plant or its surroundings. In a further form, the composition is mixed with a diluent such as water to form a spray, foam, gel or dip and applied appropriately using known protocols. In a presently preferred embodiment, a B. bassiana composition formulated as described above is mixed with water using a pressurised sprayer at about 1 gm/L, or about 1 to 3 kg/ha in no less than 1000 L water per ha. Preferably, Fortune Plus™ is added to the composition as a UV and desiccation protection agent at about 1 ml/L. Compositions comprising L. muscarium, L. longisporum, or P. fumosoroseus can be applied in a similar manner.

Compositions formulated for other methods of application such as injection, rubbing or brushing, may also be used, as indeed may any known art method. Indirect applications of the composition to the plant surroundings or environment such as soil, water, or as seed coatings are potentially possible.

As discussed above, the concentration at which the compositions comprising entomopathogenic fungi of the invention or one or more metabolites thereof are to be applied so as to be effective biological control agents may vary depending on the end use, physiological condition of the plant; type (including insect species), concentration and degree of pathogen infection; temperature, season, humidity, stage in the growing season and the age of plant; number and type of conventional insecticides or other treatments (including fungicides) being applied; and plant treatments (such as leaf plucking and pruning).

For example, in certain applications, a composition comprising B. bassiana may be applied, at a rate of from about 1×10¹⁰ to about 1×10¹⁵ spores per hectare, preferably from about 1×10¹² to about 1×10¹⁴ spores per hectare, more preferably from about 5×10¹² to about 1×10¹⁴ spores per hectare, more preferably about 1-3×10¹³ spores per hectare.

In a further aspect the present invention provides a method for controlling one or more phytopathogenic insects, the method comprising applying to a plant or its surroundings a reproductively viable form and amount of B. bassiana strain K4B3.

In one embodiment, the application is of B. bassiana strain K4B3 together with one or more other entomopathogenic fungi as described herein.

Preferably, said one or more other fungi is selected from the group consisting of Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

Again, while multiple strains of the entomopathogenic fungi of the invention with activity against one or more phytopathogenic insect species may be employed in the control process, usually three strains or less are used in the process.

Repeated applications at the same or different times in a crop cycle are also contemplated. The entomopathogenic fungi of the invention, compositions comprising the entomopathogenic fungi of the invention or one or more metabolites thereof may be applied either earlier or later in the season. This may be over flowering or during fruiting. The entomopathogenic fungi of the invention, compositions comprising the entomopathogenic fungi of the invention or one or more metabolites thereof; may also be applied immediately prior to harvest, or after harvest to rapidly colonise necrotic or senescing leaves, fruit, stems, machine harvested stalks and the like to prevent insect colonisation. The entomopathogenic fungi of the invention or compositions of the invention may also be applied to dormant plants in winter to slow insect growth on dormant tissues.

Application may be at a time before or after bud burst and before and after harvest. However, treatment preferably occurs between flowering and harvest. To increase efficacy, multiple applications (for example, 2 to 6 applications over the stages of flowering through fruiting) of the entomopathogenic fungi of the invention or a composition of the invention is preferred.

Reapplication of the entomopathogenic fungi of the invention or composition should also be considered after rain. Using insect infectivity prediction models or infection analysis data, application of the BCA can also be timed to account for insect infection risk periods.

In the presently preferred embodiments, the entomopathogenic fungi of the invention or a composition comprising same or one or more metabolites thereof is applied in a solution, for example as described above, using a pressurised sprayer. The plant parts should be lightly sprayed until just before run off. Applications may be made to any part of the plant and/or its surroundings, for example to the whole plant canopy, to the area in the canopy where the flowers and developing fruit are concentrated, or to the plant stem and/or soil, water or growth media adjacent to or surrounding the roots, tubers or the like.

Preferably the entomopathogenic fungi-comprising composition is stable. As used herein, the term “stable” refers to a composition capable of supporting reproductive viability of said fungi for several weeks, preferably about one, about two, about three, about four, preferably about five, more preferably about six months, or longer. Preferably, the composition is stable without a requirement for storage under special conditions, such as, for example, refrigeration or freezing.

The applied compositions control phytopathogenic insects. Phytopathogenic insects are responsible for many of the pre- and post-harvest diseases which attack plant parts and reduce growth rate, flowering, fruiting, production and may cause death of afflicted plants. As used herein, phytopathogenic insects include insects which are themselves plant pathogens, and insects which may act as a vector for other plant pathogens, for example, phytopathogenic fungi and bacteria. It will be appreciated that by controlling host insects which act as vectors for other phytopathogens, the incidence and/or severity of plant disease can be minimised.

Examples of the major phytopathogenic insects afflicting a number of important horticultural crops grown in New Zealand are presented in Table 1 below.

TABLE 1
Major Insect Pests

| | No. of | Planted area | |
|---|---|---|---|
| Crop | Growers | (ha) | Major Pest |
| Cherries | | 550 | Aphids |
| Potatoes | 321 | 10,611 | Aphids, whitefly |
| Tomatoes (indoor) | 390 | 167 | Whitefly, caterpillars |
| Brassicas | 227 | 3,746 | Whitefly, caterpillars |
| Squash | 181 | 6,560 | Whitefly, aphids |
| Tamarillos | 175 | 270 | Whitefly, aphids |
| Strawberries | 125 | 361 | Aphids, thrips |
| Cucumber (indoor) | | 55 | Aphids, thrips, whitefly |
| Onions | 150 | 5,488 | Thrips |
| Tomatoes (outdoor) | 80 | 609 | Whitefly, caterpillars, |
| | | | thrips |
| Capsicum | 142 | 87 | Thrips, aphids, whitefly, |
| | | | caterpillars |
| Lettuce | 252 | 1,287 | Aphids, thrips |
| Pumpkin | 125 | 1,033 | Whitefly, aphids |

Control of whitefly, thrips, aphids, and caterpillars in the crops outlined above using the compositions and method of the present invention is particularly contemplated. Control of Varroa mite using B. bassiana K4B3, either alone or together with other B. bassiana strains, or with L. muscarium, or Paecilomyces fumosoroseus and compositions of the present invention comprising same are also particularly contemplated.

The methods of the invention have particular application to plants and plant products, either pre- or post-harvest. For example, the composition of the invention may be applied to stored products of the type listed above including fruits, vegetables, cut flowers and seeds. Suitable application techniques encompass those identified above, particularly spraying.

The composition can potentially be used to treat or pretreat soils or seeds, as opposed to direct application to a plant. The composition may find use in plant processing materials such as protective coatings, boxes and wrappers.

Also encompassed by the present invention are plants, plant products, soils and seeds treated directly with an active strain of the entomopathogenic fungi of the invention or a composition of the invention.

In a further aspect, the present invention extends to the use of entomopathogenic fungi of the invention in a composition of the invention.

The invention consists in the foregoing and also envisages constructions of which the following gives examples only and in no way limit the scope thereof.

### Example 1

### Identification and Isolation of Beauveria bassiana Strain K4B3

Beauveria bassiana K4B3 was originally isolated from a group of dead cicada pupae that had come to the surface of the soil and died on mass in a pine forest at Bombay, New Zealand. Fungi was isolated from the insect sample using standard procedures, including growth at 24 C at 93% relative humidity to maximise sporulation. Individual colonies were then sub-cultured onto MEA to yield pure strains for screening for entomopathogenic efficacy.

### Beauverium Characteristics

The isolate was identified as Beauverium bassiana using taxonomic references well known in the art.

### Morphological Characteristics

The isolate K4B3 is pathogenic to thrip juveniles, adults, and pupae, aphids and whitefly. This isolate has the following identifying characteristics:

Mycelium: Grows readily on MEA. Colonies are generally white at the edge becoming cream to pale yellow. Very occasionally reddish. Underside of mycelium thallus infuses a red blush pigment into agar.
Conidiophores: Abundant, rising from hyphae. 1-2 μm wide bearing groups of clustered conidiogenous cells 3-6×3-5 μm which may branch to give rise to further conidiogenous cells, globular to flask shape with well developed stalk up to 20 μm long by 1 μm wide, geniculate with denticles up to 1 μm wide.
Conidia: Clear globose conidia that are 2-3×2-2.5 μm. Blastospores are formed in submerged culture. Hydrophobic. Produces very clumped granular aggregations on agar. The colour of the spores aggregations changes to a deep almost iridescent yellow in colour at maturity. Introduction of K4B3 into submerged culture produces an extreme red colour and an acrid metallic odor while infusing a toxic metabolite into the solution.

### Deposits

The deposits referred to herein were made at the National Measurement Institute of Australia (NMIA, formerly the Australian Government Analytical Laboratories (AGAL)), as shown. As used herein, reference to an AGAL accession or deposit number should be considered equivalent to a reference to an NMIA accession or deposit number. K4B3 was deposited at National Measurement Institute of Australia on 23 Sep. 2008, and issued deposit number V08/025855.

### Example 2

### Comparison of Whitefly Control Using K4B3 and Chemical Insecticides

### Introduction

This example describes field trials that are conducted to assess the efficacy of Beauverium bassiana strain K4B3 as a biological control agent of whitefly, and comparing same to established chemical treatment procedures. The trial is conducted in two 1680 m² Venlo style Faber glasshouses, complete with coal fired boilers and Chemtest environment and irrigation controllers. The glasshouses are in all cases, apart from drainage of runoff, identical. A chemical pesticide regime is conducted in Glasshouse 1, and a trial of the BCA of the present invention is conducted in Glasshouse 2.

### Methods

Glasshouse 1

This glasshouse is planted with the De Ruiter variety Antarctica. As is normal practice after establishment, plants are allowed to reach knee high before Vydate (240 gm/L oxamyl) is applied via the irrigation at 100 ml/1000 m². As the crop progresses, the dose of Vydate is increased to 200 ml/100 m² and finally 300 ml/1000 m² when the crop has reached shoulder height. Vydate with an LD50 of 37 mg/kg must be withdrawn within 4 weeks of harvest so as to not exceed the maximum recommended level (MRL) for oxamyl in the fruit. Normal regimes of Lannate (200 gm/L methomyl) and Thiodan (350 gm/L endosulfan) mixture, Chess (250 gm/kg pymetrozine) and Attack (25 gm/L permethrin plus 475 gm/L pirimiphos methyl) are then followed.

Glasshouse 2

This glasshouse is planted with the De Ruiter variety Toronto. This variety is a much harder variety to manage than the Antartica variety planted in Glasshouse 1. As is normal practice, Whitefly control is initially performed with Vydate and follows the same regime as described above for Glasshouse 1. On withdrawal of the Vydate, Beauverium bassiana strain K4B3 is applied. Beauverium bassiana strain K4B3 is introduced into IL of 0.1% Triton x 100, and built up to a spore concentration of 1010/ml using a haemocytometer. The spore solution is chilled to 2° C. and then transported immediately to Glasshouse 2. This spore solution is then added to the 100 L spray tank to achieve a spore count of 10⁷/ml to achieve infection threshold. Fortune Plus™, a food grade vegetable oil, is then added as a humectant at the rate of 100 ml/100 L.

Spores are applied in falling temperatures and high humidity so that the greenhouse will exceed 80% rH during the night. This process is repeated 4 times in the following month and then repeated 4 times in two months later.

### Results

Crop health and the numbers of Whitefly scale on the lower leaves of each crop is assessed. Infection rate is determined quantitatively by counting the number of whitefly scale on a representative number of plants in each glasshouse, so as to determine a whitefly scale average per plant.

### Discussion

Results indicating that plants to which the BCA have been applied are in excellent condition with noticeably less whitefly scale than those of Glasshouse No. 1 are supportive of the entomopathogenic efficacy of Beauverium bassiana strain K4B3.

Results indicating that the average number of Whitefly scale on plants treated with the BCA formulation is lower than that on plants treated with chemical insecticides support the conclusion that the Beauverium bassiana strain K4B3 provides excellent control of whitefly coupled with a simple application regimen.

### Example 3

### Production of Beauveria bassiana

### Introduction

This example describes a method for the large scale solid phase growth of Beauverium bassiana strain K4B3 and the production of a composition comprising one or more metabolites thereof.

### Methods

Germination of Conidia

Optimal spore formation requires a saturated atmosphere and a temperature of 25 to 30° C. Following spore formation, spores were transferred into a dry sealable container and stored at 8° C. The spores may be so stored for up to 635 days.

Solid Phase, Large Scale Growth

After 300 days in storage, the hydrophobic spore powder was removed from storage and its viability was tested as follows.

Malt Extract Agar (fortified with 20,000 I.U. Penicillin/L and 40 mg Streptomycin/L) at pH 5.5 was prepared. A 1 mL aliquot of a spore suspension was added to the Malt Extract Agar, smeared and incubated for 14 days at 24° C.

At 15 days, the fungi was harvested into sterile water supplemented with 0.01% Triton x 100 to a concentration of 10⁴ conidia/mL. The solution was then checked for contamination. If any contamination was present, the spore solution was discarded.

Malt Extract Agar was prepared as above and added to sterile glass bulking-up trays, which were then placed into a humidity bag, sealed and cooled to 30° C. 20 mL of spore solution was then added to the trays, which were then incubated for 14 days. The fungi was again harvested into sterile water supplemented with 0.01% Triton x 100, this time to a concentration of to 10⁶ conidia/mL. The solution was then checked for contamination. Again, if any contamination was present the spore solution was discarded.

1.6 kg of kibbled red wheat was prepared and added to growth bags (ventilated with sterile tubes) along with 320 mL sterile water. The bags were then autoclaved by microwave for 3 minutes and allowed to cool to room temperature.

320 mL of spore solution was combined with Yeast Extract (2 g/L), placed into each growth bag containing the wheat and the bags incubated at 24° C. for 7 days, under artificial lighting emulating normal photoperiod.

At day 7, the ventilation tubing was removed. The fully grown cultures were harvested by adding 3 L of sterile water supplemented with 0.01% Triton x 100 to the bags, agitating the contents, and then the contents were poured into a vat. The harvesting step was repeated once.

The resulting supernatant was filtered through a 1 μM filter to remove all Beauveria and yeast spores, and was then adjusted to pH 3.9 using citric acid.

### Results

During incubation in the bags, the mycelia of the Beauveria bassiana K4B3 became pink to pinky-red, and the wheat and condensed moisture in the bags was infused with a pink colour. Without wishing to be bound by any theory, the Applicants believe that the presence of Baker's yeast in the partially sterilized wheat begins to compete for nutrient resources, prompting the Beauveria bassiana K4B3 to secrete a biotoxin or biotoxin complex, which may be or may be associated with the pink to pinky-red coloured metabolite(s), into the media.

### Example 4

### Analysis of Beauveria Cultures for Beauvericin and Other Biotoxins

### Introduction

This example describes the analysis of the biotoxin(s) produced by Beauverium bassiana strain K4B3.

### Methods

Preparation of Beauvericin Standards

1.22 mg beauvericin powder (AnaSpec Inc. San Jose, Lot #AE6017) was mixed in methanol and made up to a volume of 10 mL. Four samples were prepared, two of which contained either high or normal methionine.

LCMS

A Waters 2698 HPLC with UV diode array detector (DAD) and a Quattro Ultima triple quadrupole mass spectrometer (Waters-Micromass Ltd, UK) were used to generate mass spectra, and to detect beauvericin and other actives present in the samples. Chromatographic separation was performed using a Phenomenex Luna C18 column (50×2 mm) with a methanol gradient elution.

Data was acquired in positive full scan mode monitoring 200-1600 amu and the DAD monitored 200-400 nm.

Preparation of Beauverium bassiana Strain K4B3 Extracts

Various samples of filtered K4B3 culture supernatants were prepared as described in Example 3 above.

### Results

The amount of beauvericin detected in each of the standard samples and that present in various Beauverium bassiana strain K4B3 extracts is shown in Table 2.

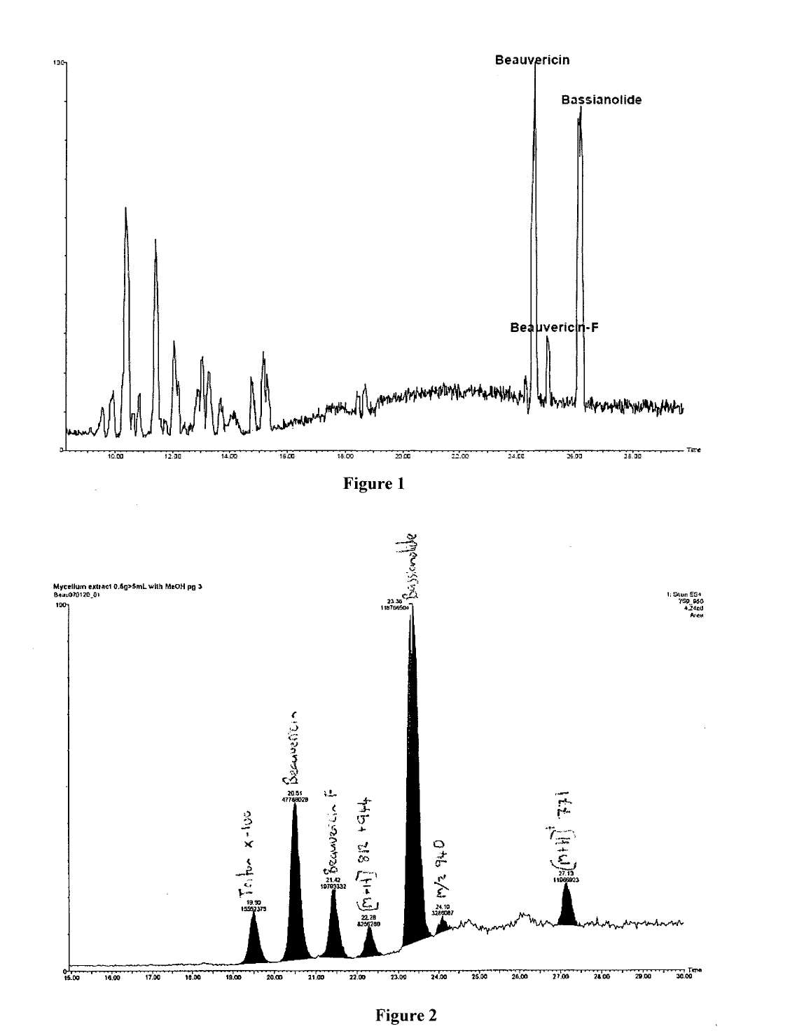

A mass spectrometric analysis of the Beauvericin—normal methionine standard was performed as described above and is shown in FIG. 1. Peaks identified as beauvericin, beauvericin-F and bassianoide were observed.

A mass spectrometric analysis of Beauverium bassiana strain K4B3 sample 3 was also performed as described above. No beauvericin was observed in the extract.

TABLE 2
Quantitation of beauvericin samples

| Sample | Beauvericin (mg/L) |
|---|---|
| Beauvericin - high methionine | 3.1 |
| Beauvericin - normal methionine | 5.9 |
| K4B3 sample 1 | 0.001 |
| K4B3 sample 2 | 0.0032 |
| K4B3 sample 3 | <0.0005 |

### Example 5

### Efficacy and Toxicity of Fungal Broth Produced by Beauveria bassiana K4B3

### Introduction

This example reports the efficacy of the K4B3 extract complex as an insecticidal agent against aphids and psyllids.

### Methods

K4B3 extract was prepared as described above in Example 3. A solution of purified beauvericin was prepared as described above in Example 3.

2 mL of K4B3 extract was applied to crops infested with aphids and psyllids. Application was initially done by hand spraying to the point of runoff (experiments reported in Tables 3 and 4 below). Subsequently, a Potters Tower was used to apply the solutions (experiments reported in Tables 5 to 7 below).

Insect morbidity was assessed at 48 and 72 hours after treatment.

### Results

K4B3 filtered broth and culture extract were active against aphids and psyllids, as shown in Tables 3 to 6 below.

TABLE 3
Adult and juvenile aphids - 48 hours after treatment.

| | | Adults | | Juveniles | |
|---|---|---|---|---|---|
| Treatment | Test | Live | Dead | Live | Dead |
| Water Control | 1 | 10 | 0 | 60 | 0 |
| | 2 | 3 | 7 | 1 | 0 |
| | 3 | 2 | 8 | 2 | 1 |
| | 4 | 10 | 0 | 58 | 0 |
| K4B3 culture broth | 1 | 0 | 10 | 0 | 0 |
| | 2 | 0 | 10 | 0 | 1 |
| | 3 | 0 | 10 | 0 | 0 |
| | 4 | 0 | 10 | 0 | 0 |
| K4B3 culture broth + stimulant | 1 | 0 | 10 | 0 | 0 |
| (1 g/L) and M oil (2.5 mL/L) | 2 | 0 | 10 | 0 | 0 |
| | 3 | 1 | 9 | 0 | 0 |
| | 4 | 1 | 9 | 0 | 0 |
| K4B3 extract + stimulant (1 g/L) and | 1 | 0 | 10 | 0 | 0 |
| M oil (2.5 mL/L) | 2 | 0 | 10 | 0 | 0 |
| | 3 | 1 | 9 | 5 | 0 |
| | 4 | 0 | 10 | 0 | 0 |
| Beauvericin (0.24 g) + 24 mL | 1 | 0 | 10 | 4 | 0 |
| stimulant (1 g/L) + M oil (2.5 mL/L) | 2 | 0 | 10 | 0 | 3 |
| | 3 | 4 | 6 | 20 | 1 |
| | 4 | 2 | 7 | 7 | 1 |

TABLE 4
Adult and juvenile psyllids - 72 hours after treatment.

| | | Larvae | | Adult | |
|---|---|---|---|---|---|
| Treatment | Test | Alive | Dead | Alive | Dead |
| Water Control | 1 | 22 | 0 | 10 | 0 |
| | 2 | 27 | 0 | | |
| | 3 | 22 | 0 | | |
| | 4 | 28 | 0 | | |
| | 5 | 21 | 0 | | |
| K4B3 extract + stimulant | 1 | 1 | 17 (1) | 0 | 5 (5) |
| (1 g/L) + M oil (2.5 mL/L) | 2 | 0 | 38 (1) | | |
| | 3 | 0 | 18 | | |
| | 4 | 0 | 12 | | |
| | 5 | 0 | 16 | | |
| K4B3 extract + K4V1 + K4V2 | 1 | 2 | 14 | 0 | 5 (4) |
| spores + stimulant (1 g/L) and | 2 | 0 | 10 | | |
| M oil (2.5 mL/L) | 3 | 0 | 12 (1) | | |
| | 4 | 0 | 10 | | |
| | 5 | 3 | 8 | | |
| K4B3 extract | 1 | 1 | 7 | 4 | 4 (4) |
| | 2 | 0 | 11 (1) | | |
| | 3 | 0 | 10 | | |
| | 4 | 0 | 14 | | |
| | 5 | 0 | 11 | | |

(n) = number of dead psyllids on which penicillium mycelium was observed Aphid mortality

Table 5 below presents mortality observed amongst green pea aphid (Myzus persicae) present on Asian brassica leaf discs. Asian brassica leaf discs were placed on 1% water agar in Petri dishes, and the dishes placed in a Potters tower. Sprays were applied with the Potter tower using 2, 5, 10, 15, and 20 mL of solution. The sprayed Petri dishes were placed dorsal side down on paper towels to allow surfaces to dry. Five replicates of each treatment were done. Mortality was assessed 24 hours after application.

TABLE 5
Aphid mortality 24 hours after treatment.

| 2 ml | | 5 ml | | 10 ml | | 15 ml | | 20 ml | |
|---|---|---|---|---|---|---|---|---|---|
| alive | dead | alive | dead | alive | dead | alive | dead | alive | dead |

Water sprayed controls

| | | | | | | | | | | |
|---|---|---|---|---|---|---|---|---|---|---|
| 1 | 10 | 0 | 11 | 0 | 9 | 0 | 10 | 0 | 8 | 0 |
| 2 | 10 | 0 | 10 | 0 | 10 | 1 | 10 | 0 | 8 | 0 |
| 3 | 6 | 0 | 10 | 0 | 9 | 0 | 7 | 1 | 9 | 0 |
| 4 | 10 | 0 | 10 | 0 | 10 | 0 | 10 | 1 | 11 | 0 |
| 5 | 10 | 0 | 10 | 0 | 11 | 0 | 10 | 0 | 8 | 1 |
| Total | 46 | 0 | 51 | 0 | 49 | 1 | 47 | 2 | 44 | 1 |
| % Mortality | | 0% | | 0% | | 2% | | 4% | | 2% |

Biotoxin complex

| | | | | | | | | | | |
|---|---|---|---|---|---|---|---|---|---|---|
| 1 | 0 | 12 | 0 | 10 | 0 | 9 | 0 | 10 | nd | nd |
| 2 | 0 | 8 | 0 | 10 | 0 | 10 | 0 | 8 | nd | nd |
| 3 | 0 | 10 | 0 | 10 | 0 | 10 | 0 | 10 | nd | nd |
| 4 | 0 | 8 | 0 | 10 | 0 | 9 | 0 | 10 | nd | nd |
| 5 | 0 | 10 | 0 | 11 | 0 | 10 | 0 | 10 | nd | nd |
| Total | 0 | 48 | 0 | 51 | 0 | 48 | 0 | 48 | | |
| % Mortality | | 100% | | 100% | | 100% | | 100% | | |

Table 6 below presents mortality observed amongst green pea aphid (Myzus persicae) present on Asian brassica leaf discs. Asian brassica leaf discs were placed on 1% water agar in Petri dishes. K4B3 extract was diluted as shown, and 2 mL of each dilution was sprayed using the Potters tower. The sprayed Petri dishes were placed dorsal side down on paper towels to allow surfaces to dry. Mortality was assessed 24 hours after application.

TABLE 6
Aphid mortality-K4B3 concentration effect.

| | Alive | Moribund | Dead | Alive | Moribund | Dead |
|---|---|---|---|---|---|---|
| control | 10 | | 0 | | | |
| Concentration 1 | 0 | | 10 | | | |
| 0.5 | 0 | | 11 | | | |
| 0.25 | 1 | 1 | 7 | | | |
| 0.125 | 4 | 2 | 2 | | | |
| 0.0625 | 7 | | 1 | 9 | | 1 |
| 0.03125 | | | | 9 | | 1 |
| 0.015625 | | | | 9 | 1 | 0 |
| 0.007813 | | | | 6 | | 1 |
| 0.003901 | | | | 7 | | 3 |
| Control | | | | 10 | | 0 |

Table 7 below presents mortality observed amongst green pea aphid (Myzus persicae) present on Asian brassica leaf discs. Asian brassica leaf discs were placed on 1% water agar in Petri dishes. 2 mL of K4B3 extract, beauvericin solution (50μg/mL, supplemented with 2.5 mL millennium oil/L), and a water control was sprayed using the Potters tower. The sprayed Petri dishes were placed dorsal side down on paper towels to allow surfaces to dry. Five replicates of each treatment were done. Mortality was assessed 48 hours after application.

TABLE 7
Aphid mortality-comparison of K4B3 and beauvericin.

| | control | | | | K4B3 extract | | | | Beauvericin | | | |
|---|---|---|---|---|---|---|---|---|---|---|---|---|
| | Alive | | Dead | | Alive | | Dead | | Alive | | Dead | |
| | Adult | Juv. | Adult | Juv. | Adult | Juv. | Adult | Juv. | Adult | Juv. | Adult | Juv. |
| Replicate 1 | 5 | 17 | 1 | 1 | 0 | 0 | 10 | 0 | 8 | 22 | 1 | 1 |
| 2 | 9 | 17 | 1 | 1 | 0 | 0 | 9 | 0 | 10 | 28 | 0 | 3 |
| 3 | 8 | 16 | 1 | 1 | 0 | 0 | 10 | 1 | 0 | 0 | 0 | 0 |
| 4 | 11 | 31 | 0 | 0 | 0 | 0 | 9 | 0 | 0 | 0 | 0 | 0 |
| 5 | 10 | 26 | 0 | 2 | 0 | 0 | 9 | 0 | 0 | 0 | 0 | 0 |
| Total | 43 | 107 | 3 | 5 | 0 | 0 | 47 | 1 | 18 | 50 | 1 | 4 |
| % Mortality | | | 7% | 4% | | | 100% | 100% | | | 5% | 7% |

### Discussion

K4B3 extract gave consistently high aphid mortality. This effect is dose dependent (as shown in Table 6). In comparison, pure beauvericin showed very low mortality, even at 50,000 μg/L. Indeed, as shown in Table 7 the mortality observed with beauvericin alone was not significantly different to that observed with the water control.

The Applicants believe, without wishing to be bound by any theory, that the low concentration of beauvericin present in the K4B3 extract (approximately 7.5 μg/L), and the comparably low concentration of bassianolide, suggests that the entomopathogenic efficacy of the K4B3 extract complex may not be due solely to either beauvericin or bassianolide. The Applicants believe, again without wishing to be bound by any theory, that the entomopathogenic efficacy may in fact be due to one or more other metabolites of K4B3 that may either have entomopathogenic efficacy themselves, or potentiate the entomopathogenic efficacy of or synergise with one or more entomopathogenic agents present in the extract.

### Example 6

### Identification of Biotoxins Present in Beauveria bassiana K4B3 Extract

### Introduction

This example describes the identification of biotoxins present in an extract produced by Beauverium bassiana strain K4B3.

### Methods

Preparation of Beauverium bassiana Extract and Identification of Biotoxins

Beauverium bassiana K4B3 extract was prepared as described in Example 3 and biotoxins detected using LCMS as described in Example 4.

### Results

Peaks identified as beauvericin, beauvericin-F and bassianoide were observed (as shown in FIG. 2). Unidentified peaks were also detected (see FIG. 2).

### Discussion

The Applicants believe, without wishing to be bound by any theory, that one or more metabolites responsible for one or more of the unidentified peaks present in the spectra may be responsible for the observed entomopathogenic efficacy of the K4B3 extract.

### Example 7

### Toxicity OF Beauveria bassiana K4B3 Extract in Mammalian Model

### Introduction

This example reports an assessment of the toxicity of the K4B3 extract complex in a mammalian model.

### Methods

K4B3 extract was prepared as described above in Example 3.

Testing was conducted in mice according to OECD Guideline 425 (Acute Oral Toxicity—Up-and-down Procedure). Since this material was not expected to be highly toxic, the Limit Test with a single dose level of 2,000 mg/kg by oral intubation was chosen. This dose is the highest recommended by the OECD for evaluation of acute toxicity, except under exceptional circumstances.

A single 2,000 mg/kg dose of K4B3 complex was administered by oral intubation to five female Swiss mice, as follows.

Test Conditions

Food was withdrawn from one of the mice at approximately 4 pm and its body weight was measured. Next morning, the mouse was weighed again and the weight of K4B3 extract required to provide a dose of 2,000 mg/kg was calculated. This amount was weighed, and diluted with 150 μl of water. The whole volume was administered to the mouse by gavage.

After dosing, the mouse was allowed immediate access to food. It was observed intensively for 60 minutes after dosing and then at several intervals throughout the day of dosing and subsequent days, as specified in the OECD Guideline for the Testing of Chemicals, Revised Draft Guideline 425, October 2000. A second mouse was dosed with K4B4 extract 48 hours after the first, again at a dose of 2,000 mg/kg body weight. The third, fourth, and fifth mice were subsequently dosed at 48 hour intervals, all at 2,000 mg/kg.

The mice were housed individually with water and food ad lib (except for the overnight fast before dosing). Mice were observed daily and body weight measured for 2 weeks following administration. Body weights were recorded 1 day, 1 week, and 2 weeks after dosing, after which the animals were killed by carbon dioxide inhalation and subjected to post-mortem examination.

### Results

No toxic effects were observed after administration of the K4B3 complex, with mice remaining in good health throughout the observation period. The mice began feeding shortly after dosing, and their behaviour during the day of dosing, and throughout the experiment, was entirely normal. No signs of diarrhoea were seen, and the faecal pellets of the mice were of normal consistency.

Body weights. The mean body weights of the mice and individual values for each mouse at various time intervals throughout the experiment are shown in Table 8.

TABLE 8
Body Weights of Mice Receiving K4B3 Extract

| | Weight before | Weight | Weight 1 | Weight 7 | Weight |
|---|---|---|---|---|---|
| | food | at | day after | days after | 14 days |
| | withdrawal | dosing | dosing | dosing | after dosing |
| | (g) | (g) | (g) | (g) | (g) |
| Mean | 25.0 | 22.6 | 24.4 | 25.3 | 25.4 |
| Mouse | | | | | |
| 1 | 25.4 | 23.1 | 25.6 | 26.5 | 25.6 |
| 2 | 25.5 | 23.2 | 24.4 | 25.8 | 27.7 |
| 3 | 27.1 | 24.1 | 25.3 | 25.6 | 26.1 |
| 4 | 23.0 | 20.8 | 22.7 | 23.8 | 23.1 |
| 5 | 24.0 | 21.6 | 24.0 | 24.6 | 24.7 |

After an overnight fast, the mice lost an average of 2.4 grams in body weight. This loss was largely regained by the next day after access to food was restored after dosing. The mice maintained their weight throughout the two-week observation period after dosing.

Post-mortem findings. No abnormalities were recorded in the mice at necropsy and the weights of the livers, kidneys, spleens, hearts, lungs and intestine (pylorus to anus) of the mice were within their normal range, as shown in Table 9.

TABLE 9
Relative Organ Weights of Mice Receiving K4B3
Relative organ weight (g/100 g body weight)

| | Liver | Kidneys | Spleen | Heart | Lungs | Intestine |
|---|---|---|---|---|---|---|
| Mean | 5.17 | 1.39 | 0.50 | 0.548 | 0.874 | 10.49 |
| Mouse 1 | 4.96 | 1.35 | 0.50 | 0.512 | 0.836 | 10.17 |
| 2 | 5.83 | 1.43 | 0.690 | 0.534 | 0.903 | 10.77 |
| 3 | 5.55 | 1.33 | 0.475 | 0.498 | 0.858 | 9.79 |
| 4 | 4.73 | 1.39 | 0.394 | 0.589 | 0.931 | 10.93 |
| 5 | 4.79 | 1.43 | 0.441 | 0.607 | 0.842 | 10.79 |

### Discussion

Oral administration of K4B3 to mice at a dose of 2,000 mg/kg caused no discernable adverse effects. No deaths occurred, and the behavior of the mice was entirely normal. No abnormalities were noted at necropsy, and organ weights were within the normal range.

The K4B3 complex exhibits low acute oral toxicity, with an LD₅₀ greater than 2,000 mg/kg body weight. This result indicates that the K4B3 complex would be classified in the lowest hazard category under the New Zealand Hazardous Substances and New Organisms (HSNO) Act 1996.

### Example 8

### Comparison of BCA Compositions

### Introduction

This example describes an assessment of the control of greenhouse whitefly achieved with the commercially available insecticidal product Mycotal™ and with a composition of the invention.

### Methods

Preparation of V+K4B3 and Mycotal™

Vertikil™, obtained from Millennium Microbes, NZ, contains conidia of Lecanicillium muscarium strains K4V1 and K4V2 and was supplied as a suspension containing 10⁹ spores/mL of each Lecanicillium strain. The provided suspension was then combined with a K4B3 biotoxin extract prepared as described in Example 3. This combined composition is referred to herein as V+K4B3. The suspension was diluted in water for spray application.

Mycotal™ (Koppert Biological Systems, Netherlands) which contains the conidia of a strain of L. muscarium was re-suspended in water for spraying.

All spray treatments were prepared according to manufacturers' instructions and applied in conjunction with appropriate adjuvants as advised. For example, Mycotal™ was applied with the oil “Addit” at a concentration of 0.25% v/v. V+K4B3 was applied with the organosilicone/vegetable oil adjuvant “Deep Fried” at a concentration of 0.25% v/v.

Insect Assays

Insect-free tomato seedlings were placed into screened cages and adult Greenhouse whitefly, Trialeurodes vaporariorum were allowed to ovipost on them for 96 hours. The adult whitefly were then removed, whereupon the seedlings were removed to whitefly-free cages and the eggs left to hatch (within 10 to 14 days). The nymphs were left for a further 14 to 21 days to develop until they reached the late third-early fourth instar stage. Leaves were then excised from the plants and the petioles places in water cyrotubes. Leaves were selected on the basis of whitefly nymph numbers, ensuring populations were not too dense, but at least 20 nymphs were present on the underside of each leaf tested.

V+K4B3 and Mycotal™ Application

Each product was applied at the following rates:
1. X=Recommended spray rate (V+K4B3 10 mL/L; Mycotal™ 1 g/L)

2. 0.5X=Half the recommended spray rate (V+K4B3 5 mL/L; Mycotal™ 0.5 g/L)
3. 0.25X=One quarter the recommended spray rate (V+K4B3 2.5 mL/L; Mycotal™ 0.25 g/L)

Suspensions were applied using a modified air-brush to ensure leaf coverage of 200 μL/leaf. Each application was replicated three times and tested concurrently. Leaves were held at 18-20° C. in vented plastic containers and insect mortality and infection was assessed 7 days after spraying.

Statistical Analysis

Mortality data was analysed by ANOVA to compare the efficacy of the two insecticide treatment products.

### Results

The mean whitefly mortality and infection levels 7 days after spray treatment are shown in Table 10. For both fungal treatments, insect mortality and infection levels increased as product concentration increased. There was significant linear trend from low to high rate for Mycotal™ (p<0.001) but not for V+K4B3. At the highest concentration of Mycotal™, 100% of dead nymphs were infected with fungus, while 85% of dead nymphs were infected with fungus from V+K4B3 treatment. Insect mortality and infection was significantly higher (p<0.001) in treated populations (for both products) compared to untreated control populations.

TABLE 10
Greenhouse whitefly mortality and infection rates

| | Mean % mortality | | Mean % infection | | % Dead infected | |
|---|---|---|---|---|---|---|
| Dose | Mycotal ™ | V + K4B3 | Mycotal ™ | V + K4B3 | Mycotal ™ | V + K4B3 |
| Control | 8 | 6 | 3 | 0 | 28 | 0 |
| X | 89 | 70 | 89 | 61 | 100 | 85 |
| 0.5X | 76 | 69 | 75 | 55 | 98 | 78 |
| 0.25X | 40 | 55 | 36 | 42 | 88 | 78 |

Product efficacy was comparable across the range of concentrations tested. Differences in mean percent mortality between the two treatments were not significant at any of the concentrations tested. V+K4B3 induced higher levels of mortality and infection when applied at lower concentrations, while Mycotal™ induced slightly higher levels of insect infection at higher concentrations. Notably, a low but statistically significant number of immature nymphs were killed but not infected at the recommended dosage rate of V+K4B3.

TABLE 11
Comparison of infection and mortality of Mycotal ™ versus V + K4B3

| | % Dead | | % Infected | | % Dead, not infected | |
|---|---|---|---|---|---|---|
| Dose | Mycotal ™ | V + K4B3 | Mycotal ™ | V + K4B3 | Mycotal ™ | V + K4B3 |
| X | 90.2 | 69.7 | 90.2 | 59.9 | 0.0 | 9.8 |
| 0.5X | 77.2 | 67.9 | 75.9 | 54.1 | 1.3 | 13.8 |
| 0.25X | 40 | 53.9 | 34.8 | 41.0 | 5.2 | 12.9 |

### Discussion

The Applicants believe, without wishing to be bound by any theory, that the significant number of dead but not infected insects observed with V+K4B3 treatment supports the view that at least some of the mortality observed with V+K4B3 is due to the presence of the K4B3 extract in the composition.

### INDUSTRIAL APPLICATION

As will be evident from the above description, the present invention provides a strain of entomopathogenic fungi and one or more metabolites thereof, together with the compositions comprising said fungi or one or more metabolites thereof, useful for the control of phytopathogenic insects. The use of such fungi and metabolites thereof in the control of phytopathogenic insects, and methods to control phytopathogenic insects, are also provided.

### PUBLICATIONS

Abbott, W. S. 1925: A method of computing the effectiveness of an insecticide. J. Econ. Entomol. 18:265-267
Anis, A. I. M.; Brennan, P. 1982 Susceptibility of different populations of glasshouse whitefly Trialeurodes vaporariorum, Westwood to a range of chemical insecticides. Faculty of General Agriculture University College of Dublin, Research report 1980-1981: 55.
Elhag, E. A.; Horn, D. J. 1983 Resistance of greenhouse whitefly (Homoptera: Aleyrodidae) to insecticides in selected Ohio greenhouses. Journal of Economic Entomology 76: 945-948.
Georghiou, G. P. 1981 The occurrence of resistance to pesticides in arthropods, an index of cases reported through 1980. FAO of UN, Rome 1981. 172 p.
Gorman, K.; Devine, G. J.; Denholm, I. 2000 Status of pesticide resistance in UK populations of glasshouse whitefly, Trialeurodes vaporariorum, and the two-spotted spider mite, Tetranychus urticae. The BCPC Conference: Pests and diseases: 1: 459-464
Grossman, J. 1994 Onion thrips. IPM Practitioner. 16(4): 12-13
Hommes, M. 1986 Insecticide resistance in greenhouse whitefly (Trialeurodes vaporariorum, Westw.) to synthetic pyrethroids. Mitteilungen aus der Biologischen Bundesanstalt fur Land-und Forstwirtschaft 232: 376.
Purvis, S. 2002 Are KCT developing resistance to chlorpyrifos. Talking thrips in citrus October 2002 issue 2: 1
Martin, N. A., Workman, P. J. 1994 Confirmation of a pesticide-resistant strain of western flower thrips in New Zealand. Proceedings of the 47th N.Z. Plant Protection conference: 144-148.
Martin, N. A. 1996. Whitefly resistance management strategy. Pp 194-203. In: Bourdot, G. W., Suckling, D. M. (eds). Pesticide Resistance: Prevention & Management., New Zealand Plant Protection Society, Lincoln, NZ.
OECD 1998: Guidelines for the Testing of Chemicals. www.oecd.org
Wardlow, L. R. 1985 Pyrethroid resistance in glasshouse whitefly (Trialeurodes vaporariorum, Westw.). Mededelingen van de Faculteit Landbouwwetenschappen, Rijksuniversiteit, Gent 50 (2b): 164-165.

1-58. (canceled)

59. A biologically pure culture of Beauveria bassiana fungus strain K4B3 on deposit at the National Measurement Institute of Australia under Accession No. V08/025855 or a culture having the identifying characteristics thereof.

60. Spores obtainable from Beauveria bassiana fungus strain K4B3 on deposit at the National Measurement Institute of Australia under Accession No. V08/025855 or a culture having the identifying characteristics thereof.

61. Use of the fungus as claimed in claim 59 or the spores as claimed in claim 60 together with at least one carrier in the preparation of a composition.

62. A method of producing a composition, the method comprising combining a reproductively viable form of Beauveria bassiana fungus strain K4B3 on deposit at the National Measurement Institute of Australia under Accession No. V08/025855 or a culture having the identifying characteristics thereof with at least one carrier.

63. The method according to claim 62 additionally comprising combining at least one fungi selected from the group consisting of Lecanicillium muscarium strain K4V1 (National Measurement Institute of Australia Accession No. NM05/44593) or a strain having the identifying characteristics thereof; Lecanicillium muscarium strain K4V2 (National Measurement Institute of Australia Accession No. NM05/44594) or a strain having the identifying characteristics thereof; Lecanicillium muscarium strain K4V4 (National Measurement Institute of Australia Accession No. NM06/00007) or a strain having the identifying characteristics thereof; Beauveria bassiana strain K4B1 (National Measurement Institute of Australia Accession No. NM05/44595) or a strain having the identifying characteristics thereof; Beauveria bassiana strain K4B2 (National Measurement Institute of Australia Accession No. NM06/00010) or a strain having the identifying characteristics thereof; Lecanicillium longisporum strain KT4L1 (National Measurement Institute of Australia Accession No. NM06/00009) or a strain having the identifying characteristics thereof; and Paecilomyces fumosoroseus strain K4P1 (National Measurement Institute of Australia Accession No. NM06/00008) or a strain having the identifying characteristics thereof.

64. The method according to claim 62 or 63 additionally comprising the step of combining one or more metabolites of Beauveria bassiana fungus strain K4B3 on deposit at the National Measurement Institute of Australia under Accession No. V08/025855 or a culture having the identifying characteristics thereof.

65. A composition which comprises Beauveria bassiana strain K4B3 on deposit at the National Measurement Institute of Australia under Accession No. V08/025855 or a culture having the identifying characteristics thereof, together with at least one carrier.

66. A composition comprising spores obtainable from Beauveria bassiana strain K4B3 on deposit at the National Measurement Institute of Australia under Accession No. V08/025855 or a culture having the identifying characteristics thereof, together with at least one carrier.

67. A composition which comprises one or more metabolites of Beauveria bassiana strain K4B3 on deposit at the National Measurement Institute of Australia under Accession No. V08/025855, or a culture having the identifying characteristics thereof and at least one carrier.

68. The composition according to claim 67, wherein the composition additionally comprises Beauveria bassiana K4B3 (V08/025855) or a strain having the identifying characteristics thereof, or spores obtainable therefrom.

69. The composition according to claim 65 wherein said composition is a stable composition capable of supporting reproductive viability of said fungi for a period greater than about two weeks.

70. A composition according to any one of claims 65 to 69 wherein said composition additionally comprises at least one strain selected from the group consisting of Lecanicillium muscarium strain K4V1 (National Measurement Institute of Australia Accession No. NM05/44593) or a strain having the identifying characteristics thereof; Lecanicillium muscarium strain K4V2 (National Measurement Institute of Australia Accession No. NM05/44594) or a strain having the identifying characteristics thereof; Lecanicillium muscarium strain K4V4 (National Measurement Institute of Australia Accession No. NM06/00007) or a strain having the identifying characteristics thereof; Beauveria bassiana strain K4B1 (National Measurement Institute of Australia Accession No. NM05/44595) or a strain having the identifying characteristics thereof; Beauveria bassiana strain K4B2 (National Measurement Institute of Australia Accession No. NM06/00010) or a strain having the identifying characteristics thereof; Lecanicillium longisporum strain KT4L1 (National Measurement Institute of Australia Accession No. NM06/00009) or a strain having the identifying characteristics thereof; and Paecilomyces fumosoroseus strain K4P1 (National Measurement Institute of Australia Accession No. NM06/00008) or a strain having the identifying characteristics thereof.

71. The use of Beauveria bassiana strain K4B3 on deposit at the National Measurement Institute of Australia under Accession No. V08/025855 or a culture having the identifying characteristics thereof, or spores obtainable therefrom, in the control one or more phytopathogenic insects.

72. The use of a composition as claimed in claim 67 in the control one or more phytopathogenic insects.

73. The use according to claim 71 or 72 wherein said one or more phytopathogenic insects is selected from the group consisting of thrips, aphids, whitefly, caterpillars, and Varroa mite.

74. A method for controlling one or more phytopathogenic insects, the method comprising applying to a plant or its surroundings a reproductively viable form and amount of Beauveria bassiana strain K4B3 on deposit at the National Measurement Institute of Australia under Accession No. V08/025855 or a culture having the identifying characteristics thereof.

75. A method for controlling one or more phytopathogenic insects, the method comprising applying to a plant or its surroundings a composition comprising
(a) Beauveria bassiana fungus strain K4B3 on deposit at the National Measurement Institute of Australia under Accession No. V08/025855 or a culture having the identifying characteristics thereof; or
(b) one or more metabolites of Beauveria bassiana strain K4B3 on deposit at the National Measurement Institute of Australia under Accession No. V08/025855 or a culture having the identifying characteristics thereof and at least one carrier; or
(c) both (a) and (b).

76. The method according to claim 74 or 75 wherein the application is of or said composition comprises at least one additional fungi selected from the group consisting of Lecanicillium muscarium strain K4V1 (National Measurement Institute of Australia Accession No. NM05/44593) or a strain having the identifying characteristics thereof; Lecanicillium muscarium strain K4V2 (National Measurement Institute of Australia Accession No. NM05/44594) or a strain having the identifying characteristics thereof; Lecanicillium muscarium strain K4V4 (National Measurement Institute of Australia Accession No. NM06/00007) or a strain having the identifying characteristics thereof; Beauveria bassiana strain K4B1 (National Measurement Institute of Australia Accession No. NM05/44595) or a strain having the identifying characteristics thereof; Beauveria bassiana strain K4B2 (National Measurement Institute of Australia Accession No. NM06/00010) or a strain having the identifying characteristics thereof; Lecanicillium longisporum strain KT4L1 (National Measurement Institute of Australia Accession No. NM06/00009) or a strain having the identifying characteristics thereof; and Paecilomyces fumosoroseus strain K4P1 (National Measurement Institute of Australia Accession No. NM06/00008) or a strain having the identifying characteristics thereof.

77. The method according to claim 74 wherein the reproductively viable form is or comprises spores.

78. A method according to claim 76 or 77 wherein said composition comprises fungal spores and said composition is applied at a rate of from about 1×10¹⁰ to about 1×10¹⁵ fungal spores per hectare.
